(19) United States
(12) Patent Application Publication
(10) Pub. No.: US 2012/0263692 A1
(43) Pub. Date: Oct. 18, 2012

(54) Engineered Xenogeneic Cells for Repair of Biological Tissue

(76) Inventor: Alicia L. Bertone, Columbus, OH (US)

(21) Appl. No.: 13/509,844

(22) PCT Filed: Nov. 15, 2010
(86) PCT No.: PCT/US10/56702

§ 371 Date: Jul. 2, 2012

Related U.S. Application Data
(60) Provisional application No. 61/281,354, filed on Nov. 16, 2009.

(51) Int. Cl. C12N 5/10; A61K 35/12

(52) U.S. Cl. 424/93.21; 435/325

(57) ABSTRACT

A transplant system and method including at least one cell from a xenogeneic donor to be transplanted into a host. The at least one cell is adapted to include a gene that causes expression of a protein that suppresses or prevents an immune response of the host to the at least one cell.

### CROSS-REFERENCE TO RELATED APPLICATIONS

This application is a U.S. national phase filing under 35 U.S.C. §371 of International Patent Application No. PCT/US2010/056702, filed Nov. 15, 2010, which claims priority to U.S. Provisional Patent Application No. 61/281,354, filed Nov. 16, 2009, the disclosures of which are hereby incorporated by reference herein in their entireties.

### FIELD OF THE INVENTION

The present invention relates generally to the treatment and repair of biological tissues, and more specifically to the transplant of genetically engineered cells to a host to regeneratively treat and repair biological tissues.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

Various demographic aging statistics demonstrate the pervasiveness of injured and inferior tissues, such as bone and cartilage, in the United States. These statistics reflect multiple categories of musculoskeletal afflictions from which individuals suffer. These categories include: (1) slow healing fractures, such as those found in soldiers, diabetics, the elderly, and individuals suffering complications due to trauma (an estimated 1,000,000 cases annually); (2) spine fusions (an estimated 300,000 surgeries annually and approximately $18 billion in annual costs due to back pain morbidity and lost work); (3) osteoporotic fractures (an estimated 704,000 cases annually); and (4) osteoarthritis (approximately $281 billion in annual costs). The last two categories, osteoporosis and osteoarthritis, frequently result in joint replacement (an estimated 877,000 surgeries annually). It is estimated that one-fourth of the United States population (i.e., over 70 million Americans) is affected by an affliction from at least one of these four categories. Further, including and in addition to these four categories, an estimated 48.3% of the U.S. population suffers from some musculoskeletal affliction, resulting in annual costs of approximately $849 billion, or 7.7% of the United States gross domestic product (GDP) [see www.boneandjointburden.org].

Thus, there is a tremendous impact on health care costs, and society in general, due to these afflictions. Consequently, there is a tremendous need for strategies to treat and repair tissues such as cartilage and bone. However, there are drawbacks with current treatment strategies. For example, articular cartilage does not have nerves or blood supply, so natural repair is minimal or inferior, often resulting in osteoarthritis. Further, delayed bone repair is common and costly. Treatment strategies for cartilage injury and delayed bone healing include surgery [debridement, drilling, bone graft, bone substitutes, osteochondral auto- or allograft transfer (OATS), autologous chondrocyte implantation (ACI), abrasion chondroplasty, or microfracture] and adjunctive therapies (Hyaluronan injection, oral nutriceuticals). Live autologous and allogeneic transplantation for cartilage repair is performed clinically in the United States [see Tuan, R. S., A second-generation ACI for focal articular cartilage defects, Arthritis Research and Therapy (2007) 9(5);109:1802; Gooding, C. R. et al., Prospective randomized study of 2 ACI techniques, Knee (2006) 13:203] and injection of autologous stem cells for bone repair is performed internationally [i.e., OSSRON® Regenerative Medical System, SEWONCELLONTECH www.rmsbio.net]. While these therapies have been described to improve healing, they have unsubstantiated efficacy and/or lack a practical application method.

Further, live cell transplantation for tissue engineering of cartilage and bone is well supported in the laboratory [Zachos, T. et al., Gene-mediated osteogenic differentiation of stem cells by BMP-2 or -6, J Orthop Res (2006) 24:1279; Evrich, D. et al., Cartilage Engineering Using a Combination of Chondrocyte-Seeded Fibrin Gels and Polyurethane Scaffolds, Tissue Engineering (2007) 13 (9): 2207; Huang, X. et al., Porous Hydrogels as Tissue-Engineering Scaffolds for 3-Dimensional in Vitro Culture of Chondrocytes, Tissue Engineering (2007) 13: 2645], but clinically, in bone, is limited to blood, bone marrow, or bone graft, and, in cartilage, is limited to large defects (i.e., those greater than 2 cm²) or failed first debridement surgeries [Tuan, R. S. A second-generation ACI for focal articular cartilage defects, Arthritis Research and Therapy (2007) 9(5);109:1802].

The two tissue transplantation procedures currently available for cartilage are (1) a periosteal patch with chondrocyte injection (autologous chondrocyte implantation—ACI), and (2) an osteochondral plug (osteochondral autograft or allograft transfer system—OATS). These procedures have yielded positive short-term results for some patients, but the long term clinical results have demonstrated unacceptable rates of failure, including painful hypertrophy of the periosteal patch and integration failure and degeneration of the chondral graft [Tuan, R. S. A second-generation ACI for focal articular cartilage defects, Arthritis Research and Therapy (2007) 9(5);109:1802; Kreuz, P. C. et al., Graft hypertrophy and ACI, Osteo &Cart (2008) 15:1339-47]. As a result, ACI has required additional surgeries in over 30% of patients [Kreuz, P. C. et. al., Graft hypertrophy and ACI, Osteo &Cart (2008) 15:1339-47], and OATS has resulted in premature degeneration of the repair cartilage and osteoarthritis. Additionally, osteochondral autografts require the patient to give up substantial amounts of normal cartilage in order to have sufficient material to fill the defects [Tuan, R. S., A second-generation ACI for focal articular cartilage defects, Arthritis Research and Therapy (2007) 9(5);109:1802].

Newer autologous tissue implantation systems are presently in clinical trials (e.g., Matrix-induced Autologous Chondrocyte Implant—MACI®—, available from Genzyme Europe) [Behrens, P. et al., MACT/MACI:5 year followup, Knee (2006) 13:194-202], but are still burdened by donor morbidity, cell amplification costs for optimal treatments, dual surgeries, and cost of case-by-case inefficient processing.

Further, use of allografts is burdened by extensive regulatory paperwork for accountability and tracking of human tissue as well as expensive screening tests and sterile donor processing under federal Good Manufacturing Practices regulation. Risk of disease transmission in fresh, and even processed, allografts is perpetual. The result is a high cost of reimbursement burdening the health care system and escalated care costs for these procedures.

Further still, the present strategies described above are all “replacement” strategies, which replace the injured or inferior tissue with alternate tissue. Many drawbacks flow from such replacement strategies, including, but not limited to, formulation of fibrocartilage, inadequate development of repair tissue, poor cell differentiation, poor bonding to surrounding articular cartilage borders, and the need for multiple surgeries in autologous chondrocyte tissue implantation; and immune responses against allografts and the drawbacks associated with the related use of immunosuppressive drugs.

In view of the many drawbacks with present replacement strategies for tissue transplant, alternate strategies are a growing need in the aging population. Possible alternate strategies include regenerative strategies. The use of pharmaceuticals (e.g., osteoclast inhibitors and statins) [Aspenberg, Bisphosphonates, P. Acta Orthop. (2009) 80:119; Tang, Q. O. et al., Statins, (2008) 17:135] and bioactive proteins (i.e., bone morphogenetic protein-2—BMP2) [Zachos, T. et al., MSC-mediated gene delivery of BMP2 in articular fracture model, Mol Ther (2007) 15:1543; Ishihara, A. et al., BMP2 acceleration of osteotomy healing,. J. Orthop. Res (2008) 26:764; Zachos, T. A. et al., Growth factors in MS damage, Am J Vet Res (2005) 66:764; Visser, R. et al., BMP-2/collagen and bone formation, Biomaterials (2009) 30(11):2032-7] are recognized advancements in repair of bone. Further, the use of living cells in tissue repair offers additional benefits, including: (1) the option for molecular engineering to release paracrine and autocrine bioactive factors [Zachos, T. A. et al., Growth factors in MS damage, Am J Vet Res (2005) 66:764]; (2) direct integration of the cell into the regenerative process [Lu, Z. et al., MSCs induce clinical recovery of encephalomyelitis in mice, J Neuroimmunol (2009) 206(1-2):58-69; Wichterle, H. et al., Xenotransplantation of stem cells, Methods Mol Biol (2009) 482:171-83]; and (3) a broad diversity of medical application.

To that end, stem cells can be engineered for induction of cell differentiation toward various tissue types, and can provide the machinery to release an array of local endogenous growth factors [Zachos, T. et al., MSC-mediated gene delivery of BMP2 in articular fracture model, Mol Ther (2007) 15:1543; Zachos, T. A. et al., Growth factors in MS damage, Am J Vet Res (2005) 66:764; Lu, Z. et al., MSCs induce clinical recovery of encephalomyelitis in mice, J Neuroimmunol (2009) 206(1-2):58-69]. For example, stem cell neural precursors achieved measurable neurodegenerative disease recovery in mice and have been shown to differentiate into neural cells and glia in vivo. Further, genes transferred into cells can release recombinant proteins at sustained, physiologically relevant concentrations, with a greater local potency than exogenous delivery. And further still, mesenchymal stem cells have been engineered to induce T-cell anergy and successfully treat experimental autoimmune encephalitis [Lu, Z. et al., MSCs induce clinical recovery of encephalomyelitis in mice, J Neuroimmunol (2009) 206(1-2):58-69], and stem cell xenotransplants have been functionally integrated into neural networks in mammalian embryos [Wichterle, H. et al., Xenotransplantation of stem cells, Methods Mol Biol (2009) 482:171-83].

Thus, an engineered xenogeneic cell transplant could offer superior efficacy over current cell systems and reduce cost, time, donor morbidity, and disease risk. Such xenogeneic transplant strategies have been investigated. For example, the first rDNA product from genetically engineered animals (rhAnti-thrombin; ATryn™) was approved in 2009 by the FDA to potentially replace the need for human anti-thrombin derived from human blood plasma (see www.fda.gov/cm/geanimals.htm), and the hDAF-pig, genetically engineered to express human CD55, has successfully provided donor organs to humans and hundreds of patients successfully contain pig heart valves (see http://news.bbc.co.uk/2/hi/science/nature/1740316.stm).

However, xenogeneic sources present several obstacles. For example, discovery of latent retroviruses (ubiquitous in pigs) in transplant patients has hindered the use of the pig as a xenograft donor. Thus, while xenogeneic strategies for medical therapy remain attractive, a strategy to minimize the immune reaction is a challenge [Hale, D. A., Basic transplantation immunology, Surgical Clinics of North America (2006) 86:11-3-25]. Most strategies to control the immune response to organ transplants have significant toxicity and focus on suppression of an activated immune reaction (i.e. steroids, and inhibitors of DNA synthesis or calcineurin) [Gregory, C. R., Immunosuppressive agents, In: Kirk's Current Veterinary Therapy eds. (2009) J. D Bongura, D. C, Twedt, XIV ed, 254-259, WB Saunders Company, Philadelphia, Pa.].

In view of the above-described drawbacks with curent strategies for treatment and repair of tissues, new strategies are desirable.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

The present invention, in its various aspects, reduces and/or eliminates the drawbacks described above in current treatments for injured or inferior tissues. It does so by providing a regenerative strategy for tissue repair. Regenerative strategies for bone and cartilage repair offer significant impact by reducing morbidity of musculoskeletal diseases and their burden on the socioeconomic system, as well as reducing costs for reimbursements by the present health care system.

One aspect of the present invention provides cells that have been engineered such that, once transplanted into a host, they do not trigger an immune response in the host (they suppress an immune response in the host). Another aspect of the present invention provides methods of directly introducing these cells into a host to stimulate regeneration and repair of various tissues.

The engineered cells may be xenogeneic as compared to the cells of the host. Thus, various embodiments of the present invention include the use of cells obtained from xenogeneic sources in the regenerative strategies of the present invention. These xenogeneic cell sources streamline the regenerative strategies. For example, xenogeneic sources provide a greater quantity of available cells (i.e., more animals can be used as donors than by relying on human sources), and harvesting cells from a xenogeneic donor avoids other drawbacks in harvesting cells from donor humans, which is a more involved, regulated, and expensive process. Thus, use of xenogeneic cell sources results in greater efficiency—producing an effective therapy at lower cost. By providing enhanced treatment strategies at a lower cost, the present invention reduces cost of patient hospitalization, dependent living, lost work, and morbidity that limit quality of life (estimated in hundreds of billions of dollars).

However, as described above, and as is well known to those of ordinary skill in the art, a concern with any transplant is rejection by the host. Thus, as described above, one aspect of the present invention provides an engineered cell that, once transplanted, does not trigger an immune response in the host (they suppress an immune response in the host). In “not triggering” or “suppressing” the immune response, it will be understood by those of ordinary skill in the art that this does not require that the immune system of the host lies absolutely dormant, but merely that any activity of the immune system with respect to the transplanted cells does not rise to a level that prevents, suppresses, or negates the regeneration of tissue (e.g., a level of activity that would be seen with non-engineered xenogeneic cells). In other words, a state of immunotolerance (cell anergy) is provided. In particular, in embodiments of the present invention, the cells (e.g., xenogeneic cells) may be engineered to express at least one gene (i.e., a gene or genes) that serves to suppress or prevent the stimulation of an immune response by the host (once the cell or cells are transplanted into the host). Many such candidate genes are known to those of ordinary skill in the art including, but not limited to, genes for the expression of PDL1, BMP2, and CTLA4. Further, those of ordinary skill in the art will recognize that additional candidate genes may be identified in the future, and one of ordinary skill in the art could use the aspects of the present invention described herein to engineer cells with such additional candidate genes.

Thus, genes associated with a natural mechanism of the body may be used to provide and sustain immunotolerance. As an example, in one embodiment, this mechanism may involve the PD-1/PDL1 pathway. Programmed death-1 (PD-1) and its interacting ligand, programmed death ligand-1 (PDL1), co-signal for T-cell anergy, allotolerance, prevent autoimmunity, and prolong transplant cell survival [Keir, M. E. et al., PD-1 and Its Ligands in Tolerance and Immunity, Annu Rev Immunol (2008) 26:677; Tanaka, K. et al., PDL1 Is Required for Peripheral Transplantation Tolerance and Protection from Chronic Allograft Rejection, J Immunol (2007) 179(8):5204]. Recently, genetic over-expression of PDL1 on allogeneic transplanted pancreatic islet cells has been used to prevent cell rejection and serve as a therapy for experimental diabetes [Wen, X. et al., Transplantation of NIT-1 Cells Expressing PD-L1 for Treatment of Streptozotocin-Induced Diabetes, Transplantation (2008) 86(11):1596]. This pathway of immunotolerance may be used, in this embodiment of the present invention, to suppress or prevent immune reaction of human immune cells to xenogeneic cell sources as a regenerative strategy for tissues such as bone and cartilage.

More specifically, embodiments of this aspect of the present invention quantify and profile the immunoactivation and engineered-cell-induced immunotolerance of human immune cells to xenogeneic cells transplanted for tissue regeneration. Thus, for example, in the exemplary embodiment described above (cells engineered to express PDL1) this aspect of the present invention quantifies and profiles the immunoactivation and PDL1-induced immunotolerance of human immune cells to xenogeneic cell transplantation sources for tissue regeneration. To that end, and as will be described in greater detail in the Example, human splenocytes will be co-cultured with PDL1-expressing (PDL1+) or PDL1-nonexpressing (PDL1−) live xenogeneic cell sources, including mesenchymal stem/stromal cells (MSC), chondrocytes (C), and dermal fibroblasts (Dfb) to demonstrate that (1) xenogeneic MSC, C, and Dfb will invoke human T-cell activation and T-effector cell killing with the following orders of magnitude: Dfb>C>MSC, and (2) culture of human cells with xenogeneic cells expressing PDL1 will prevent T-cell activation and T-effector cell killing, specifically reduce production of proinflammatory cytokines (i.e., interleukins (IL)—IL-6, IL-8, and IL-12; and tumor necrosis factor (TNF)—TNF-α), which support the development of CD4+ Th1 cells and CD8+ cytotoxic T lymphocytes (CTL). Further, xenogeneic cells expressing PDL1 will increase secretion of anti-inflammatory cytokines (i.e., IL-4, IL-10), which interfere with up-regulation of costimulatory molecules and production of IL-12, subsequently limiting the ability of antigen presenting cells (i.e., macrophage, dendritic cells and B lymphocytes) to initiate Th1 responses and cell-mediated immunity. Again, it will be recognized by those of ordinary skill in the art that the particular cell types described above (MSC, C, and Dfb) are merely examples, and do not limit the use of other cell types.

Another aspect of the present invention quantifies the acceleration of functional bone tissue regeneration by xenogeneic transplant cells. For example, the tissue to be regenerated may be bone and/or cartilage. And, for example, in the exemplary embodiment including cells engineered to express PDL1, rats that are chimeric for human immune cells will have an articular fracture injected with controls or xenogeneic cells, with or without PDL1 expression. Bone and cartilage regeneration will be quantified by micro-computed tomography and histomorphometry. Ex vivo rat synovium culture will quantify human T-cell activation and T-effector cell killing. Thus, this aspect will demonstrate that (1) xenogeneic cells engineered to express PDL1 (PDL1+ xenogeneic cells) have equivalent rapid and complete regeneration of bone and cartilage as control syngeneic cells in a NIH mu athymic nude rat osteotomy model, and (2) engineered PDL1− xenogeneic cells significantly accelerate osteotomy healing compared to saline indicating a biologic benefit, despite a detectable immune reaction, and a threshold of clinical tolerance to xenogeneic cells.

Thus, these regenerative strategies for tissue repair will offer significant impact by reducing morbidity of diseases (such as musculoskeletal diseases) and the burden of these diseases on the socioeconomic system, as well as reduce costs for reimbursements by the health care system.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Various features, aspects, and advantages of the present invention will become better understood when the following detailed description is read with reference to the accompanying figures in which like characters represent like parts throughout the figures, wherein:

FIG. 1 is a schematic showing cultured human splenocytes being exposed to equine xenogeneic cells (at top left); T-cell activation being anticipated (at middle left); followed by T-cell killing of donor cells on second exposure (at bottom left); and expression of PDL1 on xenogeneic cells is anticipated to bond to PD-1 naturally expressed on human T-cells (at top right) to invoke a state of immunotolerance or anergy (at bottom right).

FIG. 2 is a timeline showing time course and outcomes for the process of quantifying and profiling the immunoactivation and PDL-1-induced immunotolerance of human immune cells to xenogeneic cell transplantation sources for bone and cartilage regeneration.

FIG. 3 includes photographs showing that AAV-PDL1 injected IV in mice produced immunotolerance to Ad-GFP (adenovirus expressing green fluorescent protein) expression in the liver.

FIG. 4 is a graph showing bone morphogenic protein-2 (BMP2) production by equine chondrocytes transduced with varying multiplicities of infection (MOI) of adeno-associated viral vector serotype 2 (AAV2) or scAAV2.

FIG. 5 is a schematic showing immunosuppressed rats, chimeric for human splenocytes having an articular fracture injected with equine cells expressing human bone morphogenic protein-2 (hBMP2) and/or PDL1 to define the bone and cartilage regenerative response and the human immune response.

FIG. 6 is a timeline showing time course and outcomes for quantifying the acceleration of functional bone and cartilage regeneration by xenogeneic transplant cells.

FIG. 7 are micro-computed tomography (pCT) images of healed fractures injected 14 days earlier with syngeneic MSC-BMP2 cell vectors.

FIGS. 8a and 8b show the articular edge of an osteotomy, and that syngeneic MSC-hBMP2 formed hyaline cartilage (FIG. 8b) rather than fibrous tissue (FIG. 8a).

FIG. 9 shows a rat osteotomy (left knee) injected with 5×10⁶ syngeneic luciferase+MSC.

FIG. 10 shows a paw print and digital readout (bars) of a guinea pig with right-sided osteoarthritis showing less duration and magnitude of load R.

FIG. 11 is a photograph of a western blot showing that equine cells can be engineered by an AAV vector to express high quantities of PDL-1 at levels similar to a known human cell line (HEK293).

FIG. 12 is a graph showing the proliferation of splenocytes co-cultured with equine mesochymal stem cells (EqMSC).

FIG. 13 is a graph showing the production, or lack of production, of the inflammatory cytokine IL-12 by immune cells when exposed to various stimulus (e.g., a known stimulus such as LPS, and by co-culture with xenogeneic cells).

FIG. 14 is a graph showing the production, or lack of production, of the inflammatory cytokine IL-6 by immune cells when exposed to various stimulus (e.g., a known stimulus such as LPS, and by co-culture with xenogeneic cells).

FIG. 15 is a graph showing proliferation of immune cells based on a carboxyfluorescein diacetate succinimidyl ester (“CSFE”) assay, and showing human PDL-1 expressed on the surface of the equine mesochymal stem cells suppresses the stimulation of murine immune cells.

FIG. 16A is a graph showing co-culture of murine splenocytes with equine cells (stem cells or fibroblasts) as prolonging the life span of immune cells (CD3+, CD8+, CD4+).

FIG. 16B is a graph showing co-culture of murine splenocytes with equine cells (stem cells or fibroblasts) as prolonging the life span of splenocytes in culture.

FIG. 16C is a graph showing co-culture of murine splenocytes with equine cells (stem cells or fibroblasts) as prolonging the life span of immune cells (CD3+, CD8+, CD4+).

FIG. 16D is a graph showing co-culture of murine splenocytes with equine cells (stem cells or fibroblasts), and demonstrating that equine stem cells appear to have a more profound effect on prolonging the life span of murine splenocytes than equine fibroblasts.

FIG. 16E is a graph showing co-culture of murine splenocytes with equine cells (stem cells or fibroblasts) as prolonging the life span of immune cells (CD3+, CD8+, CD4+).

FIG. 16F is a graph showing co-culture of murine splenocytes with equine cells (stem cells or fibroblasts) as prolonging the life span of splenocytes in culture.s

FIG. 17 is a graph showing human splenocytes co-cultured with xenogeneic equine cells and demonstrating that the co-culture stimulates an increase in immune cells.

### DETAILED DESCRIPTION OF THE INVENTION

One or more specific embodiments of the present invention will be described below. In an effort to provide a concise description of these embodiments, not all features of an actual implementation may be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

As described above, regenerative strategies for bone and cartilage repair offer significant impact by reducing morbidity of musculoskeletal diseases and their burden on the socioeconomic system, as well as reducing costs for reimbursements by the present health care system. By providing enhanced treatment strategies, the present invention reduces cost of patient hospitalization, dependent living, lost work, and morbidity that limit quality of life (estimated in hundreds of billions of dollars). Thus, the social and economic impact of strategies for treating tissues, such as bone and cartilage is large, and developing such strategies is of great importance. The present invention will move this strategy forward as a viable therapy for the effective use of transplanted, molecularly engineered xenogeneic cells for the purpose of bone and cartilage repair.

One aspect of the present invention provides cells that have been engineered such that, once transplanted into a host, they do not trigger an immune response in the host (they suppress an immune response in the host). Another aspect of the present invention provides methods of directly introducing these cells into a host to stimulate regeneration and repair of various tissues.

The engineered cells may be xenogeneic as compared to the cells of the host. Thus, various embodiments of the present invention include the use of cells obtained from xenogeneic sources in the regenerative strategies of the present invention. These xenogeneic cell sources streamline the regenerative strategies. For example, xenogeneic sources provide a greater quantity of available cells (i.e., more animals can be used as donors than by relying on human sources), and harvesting cells from a xenogeneic donor avoids other drawbacks in harvesting cells from donor humans, which is a more involved, regulated, and expensive process. Thus, use of xenogeneic cell sources results in greater efficiency—producing an effective therapy at lower cost. By providing enhanced treatment strategies at a lower cost, the present invention reduces cost of patient hospitalization, dependent living, lost work, and morbidity that limit quality of life (estimated in hundreds of billions of dollars).

In one embodiment, equine cells (eq, eqMSC, eqC, and eqDfb) are used as the xenotransplanted cells. These cells are used in this embodiment because they have no known species' specific transmissible virus or prion pathogen. Further, the equine retrovirus has been eliminated from the United States equine population by an extensive governmentally regulated “test and slaughter” policy that has been in existence for decades. And, an added benefit is that the equine species has been a popular model for bone and cartilage repair in the scientific arena, and is a preferred model for FDA regulatory studies. As will be recognized by those of ordinary skill in the art, any xenogeneic cell sources may be used; equine cells are merely an example.

The xenogeneic cell sources may include: bone marrow mesenchymal stem/stromal cells (MSC); chondrocytes (C); and dermal fibroblasts (Dfb) [Zachos, T. et al., MSC-mediated gene delivery of BMP2 in articular fracture model, Mol Ther (2007) 15:1543; Ishihara, A. et al., Evaluation of equine chondrocytes, synovial cells, and stem cells (to) adenovirus 5 vectors for gene delivery, Am J Vet Res (2006) 67(7):1145].

However, as described above, and as is well known to those of ordinary skill in the art, a concern with any transplant is rejection by the host. Thus, as described above, one aspect of the present invention provides an engineered cell that, once transplanted, does not trigger an immune response in the host (they suppress an immune response in the host). In “not triggering” or “suppressing” the immune response, it will be understood by those of ordinary skill in the art that this does not require that the immune system of the host lies absolutely dormant, but merely that any activity of the immune system with respect to the transplanted cells does not rise to a level that prevents, suppresses, or negates the regeneration of tissue (e.g., a level of activity that would be seen with non-engineered xenogeneic cells). In other words, a state of immunotolerance (cell anergy) is provided. In particular, in embodiments of the present invention, the cells (e.g., xenogeneic cells) may be engineered to express at least one gene (i.e., a gene or genes) that serves to suppress or prevent the stimulation of an immune response by the host (once the cell or cells are transplanted into the host). Many such candidate genes are known to those of ordinary skill in the art including, but not limited to, genes for the expression of PDL1, BMP2, and CTLA4. Further, those of ordinary skill in the art will recognize that additional candidate genes may be identified in the future, and one of ordinary skill in the art could use the aspects of the present invention described herein to engineer cells with such additional candidate genes.

Thus, genes associated with a natural mechanism of the body may be used to provide and sustain immunotolerance. As an example, in one embodiment, this mechanism may involve the PD-1/PDL1 pathway. Programmed death-1 (PD-1) and its interacting ligand, programmed death ligand-1 (PDL1), co-signal for T-cell anergy, allotolerance, prevent autoimmunity, and prolong transplant cell survival [Keir, M. E. et al., PD-1 and Its Ligands in Tolerance and Immunity, Annu Rev Immunol (2008) 26:677; Tanaka, K. et al., PDL1 Is Required for Peripheral Transplantation Tolerance and Protection from Chronic Allograft Rejection, J Immunol (2007) 179(8):5204]. Recently, genetic over-expression of PDL1 on allogeneic transplanted pancreatic islet cells has been used to prevent cell rejection and serve as a therapy for experimental diabetes [Wen, X. et al., Transplantation of NIT-1 Cells Expressing PD-L1 for Treatment of Streptozotocin-Induced Diabetes, Transplantation (2008) 86(11):1596]. This pathway of immunotolerance may be used, in this embodiment of the present invention, to suppress or prevent immune reaction of human immune cells to xenogeneic cell sources as a regenerative strategy for tissues such as bone and cartilage.

More specifically, embodiments of this aspect of the present invention quantify and profile the immunoactivation and PDL1-induced immunotolerance of human immune cells to xenogeneic cell transplantation sources for tissue regeneration. To that end, and as will be described in greater detail in the Example, human splenocytes will be co-cultured with PDL1-expressing (PDL1+) or PDL1-nonexpressing (PDL1−) live xenogeneic cell sources, including mesenchymal stem/stromal cells (MSC), chondrocytes (C), and dermal fibroblasts (Dfb) to demonstrate that (1) xenogeneic MSC, C, and Dfb will invoke human T-cell activation and T-effector cell killing with the following orders of magnitude: Dfb>C>MSC, and (2) culture of human cells with xenogeneic cells expressing PDL1 will prevent T-cell activation and T-effector cell killing, specifically reduce production of proinflammatory cytokines (i.e., interleukins (IL)—IL-6, IL-8, and IL-12; and tumor necrosis factor (TNF)—TNF-α), which support the development of CD4+ Th1 cells and CD8+ cytotoxic T lymphocytes (CTL). Further, xenogeneic cells expressing PDL1 will increase secretion of anti-inflammatory cytokines (i.e., IL-4, IL-10), which interfere with up-regulation of costimulatory molecules and production of IL-12, subsequently limiting the ability of antigen presenting cells (i.e., macrophage, dendritic cells and B lymphocytes) to initiate Th1 responses and cell-mediated immunity. Again, it will be recognized by those of ordinary skill in the art that the particular cell types described above (MSC, C, and Dfb) are merely examples, and do not limit the use of other cell types.

Another aspect of the present invention quantifies the acceleration of functional bone tissue regeneration by xenogeneic transplant cells. For example, the tissue to be regenerated may be bone and/or cartilage. And, for example, in the exemplary embodiment including cells engineered to express PDL1, rats that are chimeric for human immune cells will have an articular fracture injected with controls or xenogeneic cells, with or without PDL1 expression. Bone and cartilage regeneration will be quantified by micro-computed tomography and histomorphometry. Ex vivo rat synovium culture will quantify human T-cell activation and T-effector cell killing. Thus, this aspect will demonstrate that (1) xenogeneic cells engineered to express PDL1 (PDL1+ xenogeneic cells) have equivalent rapid and complete regeneration of bone and cartilage as control syngeneic cells in a NIH mu athymic nude rat osteotomy model, and (2) engineered PDL1− xenogeneic cells significantly accelerate osteotomy healing compared to saline indicating a biologic benefit, despite a detectable immune reaction, and a threshold of clinical tolerance to xenogeneic cells.

Thus, these aspects of the present invention involve the response of human immune cells to cell sources from xenogeneic donors for transplantation, either as untreated cells or cells genetically engineered for immunotolerance by expressing, for example, PDL1. The osteogenic efficacy of xenogeneic cells expressing PDL1 can be evaluated both in vitro and in vivo using an animal model of bone and cartilage regeneration [Zachos, T. et al., Gene-mediated osteogenic differentiation of stem cells by BMP-2 or -6, J Orthop Res (2006) 24:1279; Zachos, T. et al., MSC-mediated gene delivery of BMP2 in articular fracture model, Mol Ther (2007) 15:1543].

Further, these aspects of the present invention demonstrate that expression of, for example, PDL1 can prevent acute immune reactions to xenogeneic cell transplants, and immune regulation of transplant cell delayed rejection. The various aspects of the present invention can also be used to measure clinically relevant regeneration of bone and cartilage to quantify the effectiveness of the xenogeneic cell transplants expressing BMP2 to regenerate bone and cartilage. This is also possible even in the event of a moderate immune reaction. Quantification of effector cell (cytotoxic T-killer cell) killing initially of donor cells as well as upon repeat exposure to donor xenogeneic cells can be performed ex vivo. The various aspects of the present invention will be described in greater detail with respect to the following Example.

While the Example is directed to cells engineered to express PDL1, those of skill in the art will recognize that the techniques used in this Example are not limited to PDL1, but may be used to engineer cells for expression other than PDL1. The various techniques described herein (e.g., cell culturing, transducing, etc.) are well known to those of ordinary skill in the art, and so are not limited solely to engineering cells to express PDL1. In the same manner, while the Example is directed to cells from an equine source, and is also directed particularly to mesenchymal stem/stromal cells, dermal fibroblasts, and chondrocytes, those skilled in the art will recognize that the invention is not so limited, as different cells from different donor organisms may be used.

### Examples

### Example 1

This prophetic Example will first investigate the response of human immune cells to cell sources from xenogeneic donors. The cell sources investigated will include (1) untreated cells, and (2) cells genetically engineered to express PDL1. This Example will also investigate and evaluate the osteogenic efficacy of xenogeneic cells expressing PDL1 (both in vitro and in vivo), using an animal model of bone and cartilage regeneration [as described in Zachos, T. et al., Gene-mediated osteogenic differentiation of stem cells by BMP-2 or -6, J Orthop Res (2006) 24:1279; Zachos, T. et al., MSC-mediated gene delivery of BMP2 in articular fracture model, Mol Ther (2007) 15:1543].

Further, this Example will use equine cells as the donor cells in the cell xenotransplant model. Equine cells will be used because, as described above, (1) they have no known species' specific transmissible virus or prion pathogen; (2) the equine retrovirus has been eliminated from the United States equine population by an extensive governmentally regulated “test and slaughter” policy that has been in existence for decades; and (3) the equine species has been a popular model for bone and cartilage repair in the scientific arena, and is a preferred model for FDA regulatory studies.

The xenogeneic cell sources (i.e., the equine cells) screened in vitro will include bone marrow mesenchymal stem/stromal cells (MSC); chondrocytes (C); and dermal fibroblasts (Dfb). These will be used to screen an array of cell types with known ability for osteogenesis and chondrogenesis. [Zachos, T. et al., MSC-mediated gene delivery of BMP2 in articular fracture model, Mol Ther (2007) 15:1543; Ishihara, A. et al., Evaluation of equine chondrocytes, synovial cells, and stem cells (to) adenovirus 5 vectors for gene delivery, Am J Vet Res (2006) 67(7):1145].

More specifically, this Example is divided into two general aims: Aim 1 and Aim 2. Aim 1 will focus in part on the relative magnitude of proinflammatory versus anti-inflammatory responses of human cells to xenogeneic equine cells and the development of cell-mediated immunity versus tolerance. Further, Aim 1 will compare osteogenic differentiation in human spleen cells co-cultured with PDL1+ or PDL1− equine cells expressing hBMP2.

In Aim 2, an in vivo model of articular fracture will be used to evaluate bone and cartilage regeneration rate and quality, after transplantation of xenogeneic equine cells expressing hBMP2 with or without PDL1. The model will include an immunodepressed rat chimeric for human immune cells. This will allow assessment of human immune cell responses to xenogeneic equine cells expressing PDL1, in vivo.

Aim 1: Quantify the Magnitude and Profile of Immunoactivation and PDL1-Induced Immunotolerance of Human Immune Cells to Several Xenogeneic Cell Transplantation Sources for Bone and Cartilage Regeneration.

As described above, Aim 1 of this Example is designed to determine the order of magnitude that live xenogeneic cells will invoke human T-cell activation and T-effector cell killing. The cells to be tested will be mesenchymal stem/stromal cells (MSC), chondrocytes (C), and dermal fibroblasts (Dfb). Aim 1 is also designed to determine that live xenogeneic cells (MSC, C, and Dfb) engineered to express surface PDL1 will not invoke human T-cell activation and T-effector cell killing, indicating a tolerized or anergic donor/recipient relationship.

Thus, three tissue sources commonly used in bone and cartilage repair (bone marrow, articular cartilage, and skin) are selected, in order to rank donor cell sources (MSC, C, or Dfb) from least to greatest in terms of human immunoactivation and immunotolerance induced by PDL1. MSC and C are considered immunoprivileged, as is known to those of ordinary skill in the art, due to receptor immaturity and site privilege, respectively. The cell source determined to stimulate the least human immunoactivation and greatest immunotolerance will be the optimal xenogeneic cell type for further in vivo study (as in Aim 2).

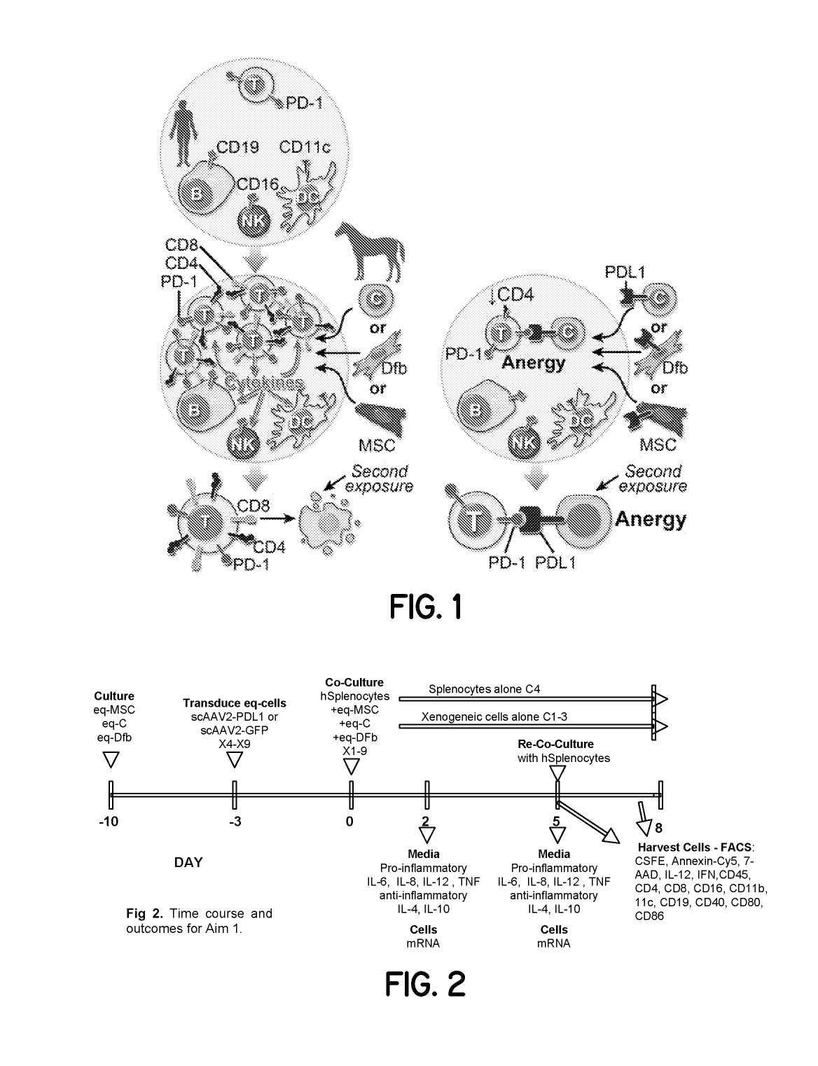

Thus, in vitro experiments will be used to match an individual human spleen to individual equine MSC, C, and Dfb for co-culture comparisons. FIG. 1 shows a schematic of this process, wherein cultured human splenocytes will be exposed to xenogeneic (equine) cells. Sources of fresh, sterile tissue include the Lifeline of Ohio Organ Donor Program (LOOP; Columbus, Ohio) for human spleens (no restrictions except negative serology) and horses aged 3-15 yrs euthanized at the Ohio State University Veterinary Teaching Hospital (Columbus, Ohio) for reasons unrelated to illness or musculoskeletal disease.

Using T-cell proliferation (as determined by CD4+ and CD8+ cell numbers by fluorescence-activated cell sorting—FACS) and xenogeneic cell death (CD45− and 7AAD+ cell numbers) as the screening parameters on Day 5 of culture, an optimal or “best” xenogeneic animal will be selected for the full study in Aim 1. The “best” xenogeneic animal will be defined as one inducing no more than modest immunoactivation of human splenocytes and xenogeneic cell death within five days of first exposure. This “best” xenogeneic animal will be selected for further comparison in up to 10 human splenocyte donors to mimic potential therapeutic application to various humans and minimize xenogeneic variability. As described above, in this Example, equine cells will be as the donor cells in the cell xenotransplant model.

Additionally, only human splenocytes that result in T-cell proliferation and cytokine production in response to LPS, anti-CD3 and anti-CD28 will be used. Should no acceptable splenocytes or matches be found within 10 spleen harvests, alternative sources for human immune cells will be evaluated, such as peripheral blood mononuclear cells (PBMCs; available from the Red Cross). Human splenocytes will be initially selected due to the ready source (LOOP and transplant operating rooms), abundant tissue source of large reservoirs of cells needed for this Example, and the possibility that the spleen (having served as an end-organ of immunity) will have a cell mixture with a more biological microenvironment.

In animals, full-thickness articular cartilage will be obtained from the knee joint, bone marrow from the distal femur, and skin (dermis) from the tibia area [as described in Ishihara, A. et al., Evaluation of equine chondrocytes, synovial cells, and stem cells (to) adenovirus 5 vectors for gene delivery, Am J Vet Res (2006)67(7):1145; and Ishihara, A. et al., Bone healing enhanced by autologous dermal fibroblasts expressing BMP2, 55th ORS (2009) 840, each of which is incorporated by reference herein in its entirety].

For tissue processing and cell culture (in both Aim 1 and Aim 2 of this Example), sterile tissue [>100 gm (cc)] of spleen, articular cartilage, bone marrow or skin will be transported to a laboratory and cells will be isolated and incubated in Dulbecco's Modified Eagles Medium (DMEM; commercially available from; Gibco, Grand Island, N.Y.) supplemented with 10% fetal bovine serum, sodium penicillin at a concentration of 50 units/mL, streptomycin at a concentration of 100 units/mL, and L-glutamine at a concentration of 29.2 mg/mL (supplemented DMEM) at 37° C. and a 5% CO₂ atmosphere [Zachos, T. et al., Gene-mediated osteogenic differentiation of stem cells by BMP-2 or -6, J Orthop Res (2006) 24:1279; Ishihara, A. et al., Evaluation of equine chondrocytes, synovial cells, and stem cells (to) adenovirus 5 vectors for gene delivery, Am J Vet Res (2006) 67(7):1145; Ishihara, A. et al., Bone healing enhanced by autologous dermal fibroblasts expressing BMP2, 55th ORS (2009) 840]. The pluripotentiality of the MSC will be confirmed by culture in controlled osteogenic, chondrogenic, and adipogenic media cocktails containing dexamethasone with ascorbate, rhTGF-betal, and dexamethasone with insulin and indomethacin, respectively, as is known to those of ordinary skill in the art [Zachos, T. et al., Gene-mediated osteogenic differentiation of stem cells by BMP-2 or -6, J Orthop Res (2006) 24:1279; Ishihara, A. et al., Evaluation of equine chondrocytes, synovial cells, and stem cells (to) adenovirus 5 vectors for gene delivery, Am J Vet Res (2006) 67(7):1145]. All cells will be seeded into 24-well plates at a total of 2.2×10⁴ cells per well in supplemented DMEM and comparisons performed in quadruplicate [Zachos, T. et al., Gene-mediated osteogenic differentiation of stem cells by BMP-2 or -6, J Orthop Res (2006) 24:1279]. A set of triplicates will be averaged to represent outcomes. One well will be sacrificed for messenger ribonucleic acid (mRNA) extraction to verify cytokine expression.

Splenocyte cultures will be set up as 2.2×10⁴ splenocytes per well, and splenocyte/xenogeneic cell co-cultures will be set up as 1.1×10⁴ splenocytes mixed with 1.1×10⁴ xenogeneic cells per well, as per the protocol shown in FIG. 2. Pure splenocyte cultures will serve as an untreated control (C4) for each human individual. Splenocyte/xenogeneic cell co-cultures will serve to compare cell source (MSC, C, Dfb) or PDL1 effect for each outcome parameter (see Table 1 and FIG. 2).

TABLE 1
Culture Assignments for Cells

| | | | X4-6 Co-cultures | X7-9 Co-cultures |
|---|---|---|---|---|
| | | X1-3 Co-cultures | (Splenocytes | (Splenocytes |
| | | (Splenocytes | plus GFP | plus PDL1 |
| C1-3 Equine (eq) | C4-6 Human (h) | plus Native | engineered | engineered |
| Xenogeneic | Splenocyte | Xenogeneic | Xenogeneic | Xenogeneic |
| Controls | Controls | Cells) | Cells) | Cells) |
| eq-MSC alone | h-Splenocytes | eq- | eq-MSC- | eq-MSC- |
| | alone | MSC/Splenocyte | GFP/Splenocyte | PDL1/Splenocyte |
| eq-C alone | h-Splenocytes + | eq-C/Splenocyte | eq-C- | eq-C- |
| | LPS (acute | | GFP/Splenocyte | PDL1/Splenocyte |
| | inflammatory | | | |
| | activation) | | | |
| eq-Dfb alone | h-Splenocytes + | eq-Dfb/Splenocyte | eq-Dfb- | eq-Dfb- |
| | antiCD3 + anti- | | GFP/Splenocyte | PDL1/Splenocyte |
| | CD28 (T-cell | | | |
| | specific activation) | | | |

Additionally, native and PDL1+ cell cultures without splenocytes will be maintained and sampled in parallel to serve as background controls. All cultures will have media changed and frozen (−80° C.) for cytokine analyses, and a well of parallel cells sacrificed for mRNA extraction, on Days 2 and 5. Cells will be harvested on Day 5 for analyses (see FIG. 2).

Thus, to test whether culture of human cells with xenogeneic cells expressing PDL1 will reduce production of proinflammatory cytokines [i.e., interleukins (IL)—IL-6, IL-8, IL-12, and tumor necrosis factor (TNF)—TNF-α], which support the development of CD4+ Th1 helper lymphocytes and CD8+ cytotoxic T lymphocytes (CTL), human splenocytes will be co-cultured with PDL1-expressing (PDL1+) or PDL1-nonexpressing (PDL1−) live equine xenogeneic cells. It is anticipated that xenogeneic cells expressing PDL1 will increase secretion of anti-inflammatory cytokines (i.e., IL-4, IL-10), which interfere with up-regulation of costimulatory molecules (i.e., CD40, CD80, CD86) and production of IL-12, subsequently limiting the ability of antigen presenting cells (APC) (i.e., macrophage, dendritic cells and B lymphocytes) to initiate Th1 responses [Interferon-gamma (IFN-γ)] and cell-mediated immunity (CMI).

Aim 1a: Response of Human Splenocytes to Known T Cell and APC Stimulations.

Fresh human splenocyte mixtures [Muramatsu, K. et al., Chimerism studies for the induction of immunotolerance to allografts, J Plast Reconstr Aesthet Surg (2008) 61(9):1009-15] will be isolated from up to 10 human organ donors and culture with lipopolysaccharide (LPS; 1 μg/ml; Escherichia coli 055:B5, commercially available from Sigma Chemical Co, St Louis, Mo.); or solid phase anti-CD3 mAb (i.e. plates coated with 5 μg/ml anti-CD3 and 2 μg/ml anti-CD28 (commercially available from BD Pharmingen, Franklin Lakes, N.J.) to profile acute inflammatory and T-cell activation, respectively. This will confirm the normal response of human splenocytes to known immunoactivators. Appropriately responding human splenocytes will then be used for the remainder of this experiment. Human spleens will be obtained through the Lifeline of Ohio Organ Donor Program, in order to obtain human splenocytes. Techniques for culturing splenocytes, including human splenocytes, and screening isolated cells are well known to those of ordinary skill in the art.

Aim 1b: Splenocyte/Xenogeneic Co-Culture

After the appropriate splenocytes have been determined, up to 10 human splenocyte mixtures will be co-cultured with 3 equine cell sources (eqMSC, eqC, and eqDfb) to quantify and profile the xenogeneic/human (h) splenocyte immune response. These cell culture techniques are well established and known to those of ordinary skill in the art [as disclosed in Ishihara, A. et al., Evaluation of equine chondrocytes, synovial cells, and stem cells (to) adenovirus 5 vectors for gene delivery, Am J Vet Res (2006) 67(7):1145; and Ishihara, A. et al., Bone healing enhanced by autologous dermal fibroblasts expressing BMP2, 55th ORS (2009) 840, each of which is incorporated by reference herein in its entirety]. Pro- and anti-inflammatory cytokine expression will be determined by enzyme-linked immunosorbent assay (ELISA) and real-time reverse transcriptase quantitative polymerase chain reaction (qRT-PCR). Both of these techniques are well known to those of ordinary skill in the art. Activation and differentiation of APCs and proliferation of splenic lymphocytes will be determined by flow cytometry after staining with an appropriate fluorescent mAb, as is known to those of ordinary skill in the art. Additionally, the induction of Th1 and CTL responses will be evaluated. Thus, the ratio of human CD4+ IFNγ+cells versus CD4+ IL-10+ cells, as well as the frequency of human CD8+ IFNγ+ cells, will be analyzed. The induction of CTL will be confirmed by measuring the killing of xenogeneic equine cells (CD45 negative cells). For this purpose, after five days of culture, cells will be stained with a fluorescent labeled anti-human CD45 mAb and Annexin V-Cy5 (apoptosis) or 7-amino actinomycin D (7-AAD, death). The number of killed equine cells will be established as the percentage of CD45 negative Annexin+ or CD45 negative 7-AAD+ cells.

Potential CTL responses of human splenocytes to a second exposure to xenogeneic equine cells will also be evaluated. Here, human splenocytes will be cultured for five days with xenogeneic equine cells, and then will be added to new xenogeneic cell cultures. After three days, the number of human cells that are CD4+ IFNγ+cells, CD4+ IL-10+cells, or CD8+ IFNγ+ cells will be analyzed. The killing of xenogeneic equine cells will also be determined as described above. Thus, the human T-cell response to these potential xenogeneic cell transplant sources will be measured, and will provide potential target sites for therapeutic immunomodulation in xenotransplantation.

Aim 1c: Generation of scAAV2-RGD Preparations, AAV Transductions, and Gene Expression

Next, the three equine cell sources (eqMSC; eqC; and eqDfb) will be transduced to express human (h)PDL1 cDNA (http://www.openbiosystems.com) encoding for the 312 amino acid sequence for human programmed death ligand 1 (hPDL1). Further, adeno-associated viral vector serotype 2 (AAV2) modified to express the Arg-Gly-Asp (RGD) peptide (αvβ3integrin ligand) at the 4C capsid region, amino acid sequence 588, and driven by the cytomegalovirus (CMV) promoter will serve as the targeted delivery vector for PDL1 [AAV-2A5884C-RGD-hPDL1) or Green Fluorescent Protein (GFP) [AAV-2A5884C-RGD-GFP] (a transduction efficiency marker and vector control) [as described in Bertone, A. L. et al., RGD Modified AAV2 Vector Provides Expanded Tropism to Articular Cells, (2004) 50th ORS:1140, incorporated by reference herein in its entirety]. Then, the human splenocyte mixtures will be co-cultured with these three PDL1+ equine cell sources (eqMSC-PDL1, eqC-PDL1, and eqDfb-PDL1) to quantify and profile the splenocyte immunotolerance to the xenogeneic cells. Culture assignments for these cells are shown in Table 1 (above). Human CD45+ cell function will be assessed and compared to results of Aim 1b to quantify the tolerance of human splenocytes to these PDL1+equine cells. This will determine human T-cell specificity of PDL1 inhibitory signaling and compare the magnitude of donor cell immunotolerance induced by PDL1 compared to the immunoactivation induced by native xenogeneic cells (reduction or shift in immunoactivation in Aim 1) (See also FIGS. 1 and 2).

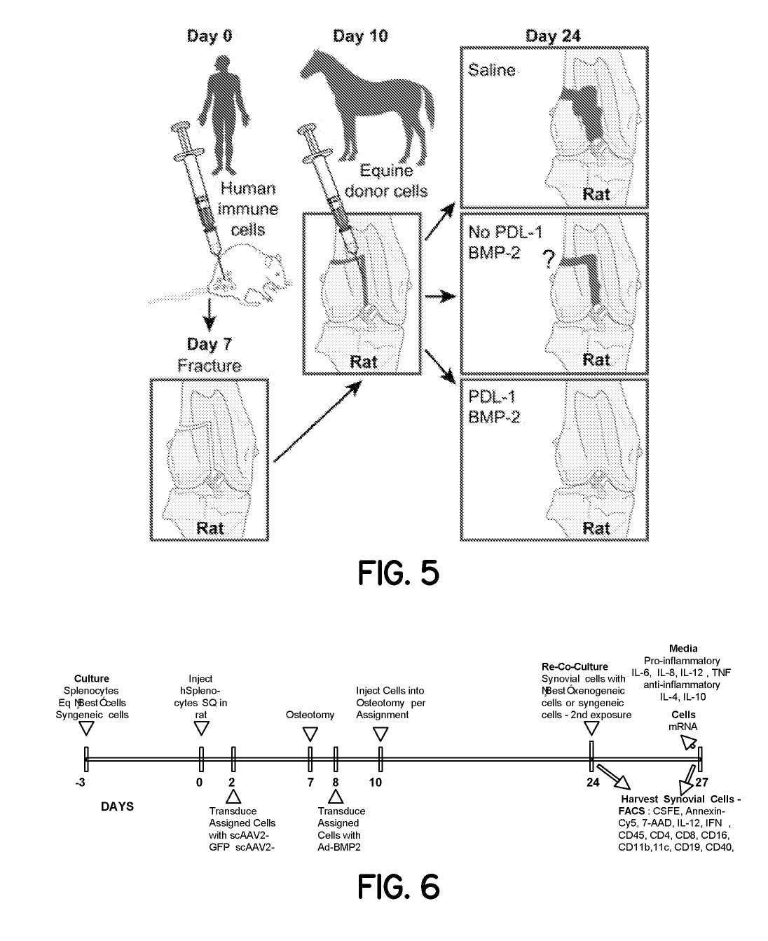

More specifically, for the generation of scAAV2-RGD preparations, AAV transductions, and gene expression, cultures and co-cultures will be established for the PDL1 assignments as shown in Table 1 and FIG. 2. A human complete PDL1 cDNA (312 AA, 936bp, Genbank Accession No. BC074984; homologue CD274) [http://www.openbiosystems.com] in a pCR4-TOPO shuttle vector will be cloned into an scAAV2-RGD plasmid containing the cytomegalovirus (CMV) promoter. Stocks of high titer scAAV2-RGD-PDL1 will be made via the three component plasmid system using (1) scAAV2-PDL1; (2) AAV2-RGD helper plasmid containing Rep and Cap genes; and (3) the Ad helper plasmid for efficient AAV genome replication and gene expression. These are standard procedures, known to those of ordinary skill in the art, such as are practiced at the Vector Core Laboratory (Nationwide Children's Hospital, Columbus, Ohio), and this system has been used to generate scAAV2-BMP2 vectors previously (see FIG. 5). These RGD-modified vectors have been developed to provide expanded tropism to many cell types [Shi, W. et al., RGD inclusion in VP3 provides AAV2-based vectors with a heparin sulfate-independent cell entry mechanism, Mol Ther (2003) 7(4):515].

Superior transduction efficiency in equine chondrocytes and MSCs by AAV-2A5884C-RGD-GFP as compared to other AAV vector options, such as standard AAV-2-GFP, AAV-5-GFP or AAV-8-GFP, has been confirmed [in Bertone, A. L. et al., RGD Modified AAV2 Vector Provides Expanded Tropism to Articular Cells, (2004) 50th ORS:1140, incorporated by reference herein in its entirety]. ScAAV mutant vectors will be chosen for use because they are a reliable and well established tool to achieve rapid onset, long-term expression [as described in McCarty, D. M., Self-complementary AAV vectors; advances and applications, Mol Ther (2008) 16:1648, incorporated by reference herein in its entirety], also shown in articular cartilage [as described in Santangelo, K., et al., Distribution of Ad and AAV2 vectors within osteoarthritic and unaffected cartilage, 55th ORS (2009) 37, 1244, incorporated by reference herein in its entirety]. For further data regarding transduction by AAV vectors, see Example 2 (below).

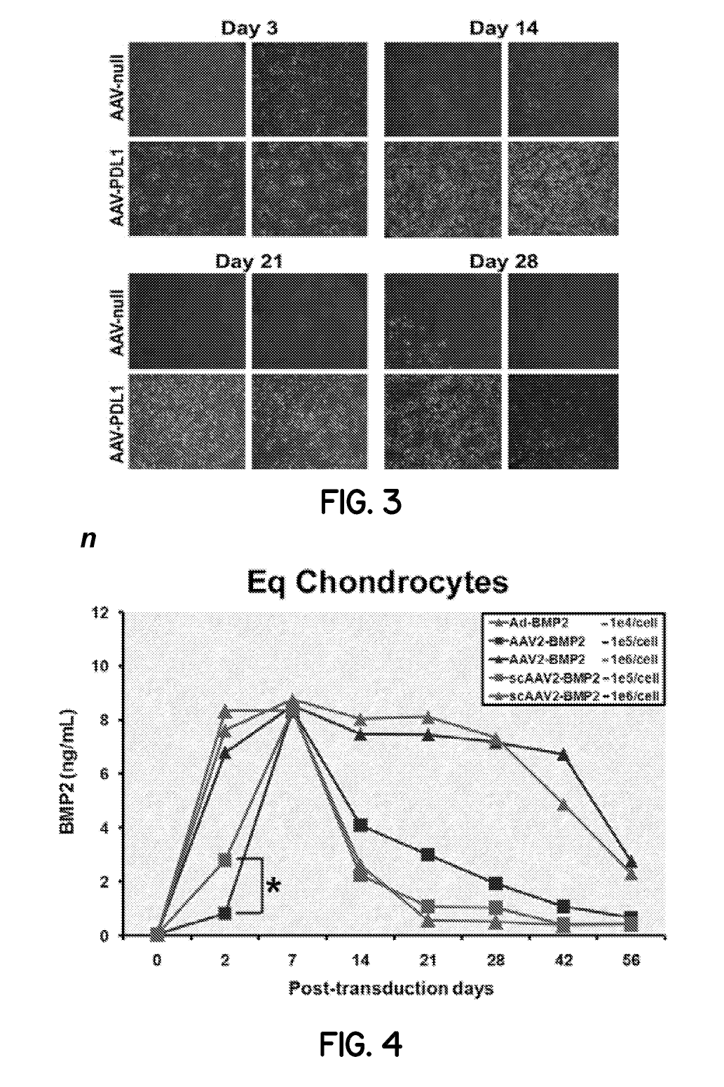

There are alternative methods for plasmid delivery, known to those of ordinary skill in the art [see Bertone, A. L. et al., Chondrogenic potential of direct plasmid gene delivery in 3-D culture, Mol Ther (2003) 7(5) Part 11:539, incorporated by reference herein in its entirety]. However, the RGD mutant scAAV vector will be used because it is a proven performer with high efficiency and sustained gene expression [see Shi, W. et al., RGD inclusion in VP3 provides AAV2-based vectors with a heparin sulfate-independent cell entry mechanism, Mol Ther (2003) 7(4):515; and McCarty, D. M., Self-complementary AAV vectors; advances and applications, Mol Ther (2008) 16:1648]. Persistent expression of PDL1 on xenogeneic cells is desired, so as to resist the human immune reaction for the duration of the study (at least 24 days), not reliably achieved with adenoviral vector [Muruve, D. A., The innate immune response to Ad vectors, Human Gene Therapy (2004) 15:1157-1166]. Additionally, recombinant (r) AAV vectors have a strong track record in basic, preclinical and clinical studies, can infect quiescent cells (unlike retrovirus), have a non-pathogenic wild-type parent virus, and limited immunogenicity [McCarty, D. M., Self-complementary AAV vectors; advances and applications, Mol Ther (2008) 16:1648]. Further, scAAV2-RGD-GFP has been prepared and PDL1-induced immunotolerance to adenovirus (Ad)-GFP expression in vivo in the liver of mice has been demonstrated, by intravenous injection of scAAV8-murPDL1 [see Nishimura, H. et al., Immunological studies on PD-1 deficient mice: implication of PD-1 as a negative regulator for B cell responses, Internal Immunology (1998) 10, 1563, incorporated by reference herein in its entirety] (see also FIG. 3).

The scAAV8-PDL1 vector codes for the full-length 290 amino acid murine cDNA. Referring to FIG. 4, a multiplicity of infection (MOI) of 1×10⁵ scAAV2 particles/cell will optimally and efficiently transduce equine chondrocytes within 48 hours and will be evaluated in the three cell sources to confirm robust GFP gene expression (>80% GFP+ cells) prior to Aims 1 and 2, as well as PDL1 expression using quantitative PCR analysis [AB Sequence Analyzer, Applied Biosystems (hPD-L1: 5′-dGCCGAAGTCATCTGGACAAG-3′ [SEQ. ID NO. 1] and 5′-dTCTCAGTGTGCTGGTCACAT-3′ [SEQ. ID NO. 2], probe 5′ FAM-dCACCACCACCAATTCCAAGA-3′ [SEQ. ID NO. 3]]. RT-qPCR for gene expression is a routine technique well known to those of ordinary skill in the art [Zachos, T. et al., MSC-mediated gene delivery of BMP2 in articular fracture model, Mol Ther (2007) 15:1543; Ishihara, A. et al., BMP2 acceleration of osteotomy healing, J. Orthop. Res (2008) 26:764; Ishihara, A. et al., Evaluation of equine chondrocytes, synovial cells, and stem cells (to) adenovirus 5 vectors for gene delivery, Am J Vet Res (2006) 67(7):1145; Ishihara, A. et al., Bone healing enhanced by autologous dermal fibroblasts expressing BMP2, 55th ORS (2009) 840] and can be performed with an Applied Biosystems Sequence Analyzer (commercially available from Applied Biosystems, Inc., Foster City, Calif.).

Cultures and co-cultures will be transduced with the “best” MOI (multiplicity of infection) of scAAV2-RGD-PDL1 or scAAV2-RGD-GFP (defined as the lowest MOI to produce >80% transduction) in quadruplicate, for fluorescent microscopic quantification of effective transduction and gene expression in Aim 1 (see FIG. 2). Outcome variables will be quantified in similar fashion to Aim 1 to assess the PDL1-induced immunotolerance of human immune cells to xenogeneic cells. It is anticipated that PDL1 will be inhibitory to T-cell activation and effector T-cell killing of xenogeneic cells. In addition to Aim 1 data, a representative well of each group in Table 1 will have PDL1 gene expression confirmed by RT-qPCR and western blot (using a working PDL1 antibody).

Aim 1d: Osteogenesis of Splenocyte/Donor Co-Cultures and Adenoviral Transduction

The xenogeneic cell source with the least human immunoactivation and greatest immunotolerance, will be determined to be the optimal, or “best”, xenogeneic transplant cell type, and will be selected to compare osteogenic differentiation of these cells in this potential state of anergy (i.e., compare osteogenic differentiation in human spleen cells co-cultured with PDL1+ or PDL1− equine cells expressing hBMP2). To that end, human splenocyte mixtures will be co-cultured with the “best” equine cell source (eqMSC, eqC, or eqDfb) and quantified for rate of appearance of positive alkaline phosphatase stain and mineralized nodules in PDL1+ compared to PDL1− co-culture in an osteogenic media cocktail [as described in Zachos, T. et al., Gene-mediated osteogenic differentiation of stem cells by BMP-2 or -6, J Orthop Res (2006) 24:1279, incorporated by reference herein in its entirety]. Additionally, osteogenic differentiation will be driven by Adenoviral (Ad) hBMP2 transduction, a potent accelerator of mineralized nodule formation and osteogenic gene expression. These osteogenic culture techniques are well established and known to those of ordinary skill in the art [as described in Zachos, T. et al., MSC-mediated gene delivery of BMP2 in articular fracture model, Mol Ther (2007) 15:1543; Ishihara, A. et al., BMP2 acceleration of osteotomy healing, J. Orthop. Res (2008) 26:764; Ishihara, A. et al., Evaluation of equine chondrocytes, synovial cells, and stem cells (to) adenovirus 5 vectors for gene delivery, Am J Vet Res (2006) 67(7):1145; and Ishihara, A. et al., Bone healing enhanced by autologous dermal fibroblasts expressing BMP2, 55th ORS (2009) 840, each of which is incorporated by reference herein in its entirety]. BMP2 production by any of these BMP2-transduced cultures will be confirmed by ELISA (Quantikine®, R&D Systems, Minneapolis, Minn.) and qRT-PCR, techniques that are well known to those of ordinary skill in the art. Mineralization of the xenogeneic donor cells is not a requirement for successful transplantation in the rat model because BMP2 secretion by the transplanted cells can accelerate bone and cartilage formation through a paracrine effect.

More specifically, osteogenesis of splenocyte/donor co-cultures and adenoviral (Ad) transduction, using established techniques well known to those of ordinary skill in the art [Zachos, T. et al., Gene-mediated osteogenic differentiation of stem cells by BMP-2 or -6, J Orthop Res (2006) 24:1279; Zachos, T. et al., MSC-mediated gene delivery of BMP2 in articular fracture model, Mol Ther (2007) 15:1543; Ishihara, A. et al., Evaluation of equine chondrocytes, synovial cells, and stem cells (to) adenovirus 5 vectors for gene delivery, Am J Vet Res (2006) 67(7):1145], the three cell sources (eqMSC, eqC, eqDfb), either as native cells or expressing PDL1, will be prepared in monolayer culture and as splenocyte co-cultures. Cells will be cultured in DMEM or DMEM with dexamethasone, ascorbate, and rhTGF-betal for 14 days to drive osteogenic differentiation. Cultures will be scored for morphology [Zachos, T. et al., Gene-mediated osteogenic differentiation of stem cells by BMP-2 or -6, J Orthop Res (2006) 24:1279], and stained for alkaline phosphatase (Sigma Kit 85, commercially available from Sigma Aldrich, St. Louis, Mo.), and mineral (vonKossa method by point counting for presence of mineralized nodules) on Days 7 and 14 to confirm osteogenic differentiation.

Similar cultures will be established and either untransduced, transduced with Ad-GFP, or transduced with Ad-hBMP2 at 37° C. for 2 hrs (multiplicity of infection (MOI) 17:1 Adeno-X™ Rapid Titer Kit, commercially available from BD Biosciences Clontech, Palo Alto, Calif.) and incubated for 14 days. Triplicate co-culture wells will be counted for GFP positive cells (Days 2, 5, 7, and 14), scored for morphology [Zachos, T. et al., Gene-mediated osteogenic differentiation of stem cells by BMP-2 or -6, J Orthop Res (2006) 24:1279; Ishihara, A. et al., BMP2 acceleration of osteotomy healing, J. Orthop. Res (2008) 26:764], and stained for alkaline phosphatase and mineral. These results will confirm the osteogenic potential and responsiveness to hBMP2 of the co-culture system and define the potential xenogeneic cell contribution to bone regeneration in vivo. Recombinant Ad vectors will contain the 1547 by open reading frame segment of human BMP2. Expression of transgenes will be verified by qRT-PCR and hBMP2 ELISA [Quantikine®, R&D Systems, Minneapolis, Minn.]. Those of ordinary skill in the art routinely propagate these vectors for large scale in vivo work [Tang, Q. O. et al., Statins (2008) 17:135; Zachos, T. et al., MSC-mediated gene delivery of BMP2 in articular fracture model, Mol Ther (2007) 15:1543; Ishihara, A. et al., Bone healing enhanced by autologous dermal fibroblasts expressing BMP2, 55th ORS (2009) 840].

For all cultured and co-cultured cells in Aim 1 (and synovium cultures from Aim 2, as will be described below), production of the secreted inflammatory cytokines, IL-6 (Cat#D6050), IL-8 (Cat#D8000C), II-12 (Cat#D1200), TNFα (Cat#DTA00C), will be quantified by ELISA [R & D Systems, Minneapolis, Minn.]. Cultured cells will be isolated by EDTA-trypsin digestion [Zachos, T. et al., Gene-mediated osteogenic differentiation of stem cells by BMP-2 or -6, J Orthop Res (2006) 24:1279; Ishihara, A. et al., Evaluation of equine chondrocytes, synovial cells, and stem cells (to) adenovirus 5 vectors for gene delivery, Am J Vet Res (2006) 67(7):1145; Ishihara, A. et al., Bone healing enhanced by autologous dermal fibroblasts expressing BMP2, 55th ORS (2009) 840], washed in phosphate buffered saline solution, stained and counted by hemacytometer for total and viable cell number by trypan blue exclusion [Ishihara, A. et al., Evaluation of equine chondrocytes, synovial cells, and stem cells (to) adenovirus 5 vectors for gene delivery, Am J Vet Res (2006) 67(7):1145], and processed by FACS Calibur Flow Cytometry (Becton Dickinson, Franklin Lakes, N.J.) for a panel of outcome variables to assess cell proliferation [Carboxy-Fluorescein Succinimidyl Ester (CSFE) assay] [Wen, X. et.al., Transplantation of NIT-1 Cells Expressing PD-L1 for Treatment of Streptozotocin-Induced Diabetes, Transplantation (2008) 86(11):1596], inflammatory activation IFNγ (Cat#DIF50), apoptosis (Annexin V-Cy5) [Wen, X. et.al., Transplantation of NIT-1 Cells Expressing PD-L1 for Treatment of Streptozotocin-Induced Diabetes, Transplantation (2008) 86(11):1596], and CD45+ cell subtyping to quantify immunoactivation and immunotolerance.

Next, the human cells, co-cultured with the xenogeneic cells, will be stained with fluorescence-labeled anti-human CD4 and anti-human CD8 mAbs (BD Pharmingen). The cells will then be fixed and intracellular staining with fluorescence-labeled anti-IFNγ will be performed with the aid of fixation and permeabilization solution as recommended by the manufacturer (BD Pharmingen). The frequency of CD4+ IFNγ+ T-cells and CD8+ IFNγ+ T-cells will be determined by flow cytometry. The number of CD8+ IFNγ+ T-cells is expected to reflect the number of cytotoxic T-cells. Quantitative RT-PCR for the analysis of cytokine/chemokine mRNA responses will be used to confirm ELISA and flow cytometry data and allow investigation of other parameters.

On cell harvest, CD4+ cell mRNA will be isolated using STAT-60 (Tel-Test, Friendswood, Tex.). Reverse transcription will be performed with Superscript II reverse transcriptase, dNTPs and poly(dT) oligos. Real-time PCR (Applied Biosystems, Foster City, Calif.) will be performed with primers generated with the PrimerExpess Software (Applied Biosystems). Results will be expressed as crossing point (CP), defined as the cycle at which the fluorescence rises appreciably above the background fluorescence as determined by the Second Derivative Maximum Method. The formula: 20−(CP_{cytokine}−CP_{-ACTIN}) will be used to represent the logarithm of the relative mRNA levels of a given cytokine. This formula allows the normalization of all results against β-actin levels to correct for differences in cDNA concentration between the starting templates.

Statistical analysis for Aim 1: Quantitative outcomes [Days 2 and 5: IL-6,IL-8,IL-12,IL-10,IL-4 for both protein and mRNA expression; Days 5 (1st exposure) and 7 (2nd exposure): CD 4+, 8+, 16+, 11b+, 11c+, 19+, 40+, 80+, 86+, CD4+/IFNγ+, CD8+/IFNγ+, and CD4+IL-10+ expressed as a ratio to CD45+ cell numbers; and Days 5 and 7: CD45−/Annexin V-Cy5, CD45− 7AAD] will be checked for normality, then analyzed using a mixed effects linear regression analysis (PROC MIXED in SAS 8.2, Cary, N.C.) for cell type (eqMSC, eqC, and eqDfb), time (repeated measures for Days 2, 5, and 7), and gene (PDL1−, PDL1+, and GFP). Splenocyte source will be included as a random effect. If humans are a significant factor, outcomes will be normalized to the individual's matching outcome from splenocyte culture alone to control for the variability among people. Using data from cytokine activations of mouse and human immune cells [Boyaka, P. N. et al., Chimeras of labile toxin one and cholera toxin retain mucosal adjuvanticity and direct Th cell subsets via their B subunit, J Immunol (2003) January 1;170(1):454; and [Boyaka, P. N. et al., Human nasopharyngeal-associated lymphoreticular tissues, Am J Pathol. (2000) 157(6):2023] (i.e., mean IL-10↑ 1893 pg/ml +/−509), a error 0.05 and a power of 0.8, an N=3 observations would detect a 50% difference in outcome. [Stata Corp, College Station, Tex.] Any less than 50% effect would be considered a clinically insufficient reduction in inflammation or cell mediated immunity. Fewer N would be needed to detect a greater difference. The study may be overpowered by using up to ten observations to be able to use more discriminating parametric analyses considering the complexity of the study design. Interim statistical analyses will be performed at 7 observations and the study may be truncated if differences are greater than 50%.

In summary, it is anticipated that the results will show that engineered expression of PDL1 on the surface of transplant cells co-cultured with human immune-cells will significantly limit T-cell function, including a decrease in CD4+ Th1/IFNγ+ cells, CD8+ CTL/IFNγ+ cells, and soluble cytokine production (IL-6, IL-8, Il-12, TNFα), as well as an increase in anti-inflammatory cytokines (IL-4, IL-10) confirming the anticipated T-cell mediation of PDL1 immunotolerance. Further, it is anticipated that CD8+cells activated by xenogeneic cells engineered to express PDL1 will demonstrate significant reduction in T-effector cell killing (fewer CD45− 7AAD+ cells and CD45− Annexin V Cy5+ cells) upon second exposure to the original (donor) cells, indicating a tolerized or anergic donor/recipient arrangement for long-term immunotolerance (as shown in FIG. 1). Reduction of T-cell mediated activation of CD11b+,11c+ (dendritic cells), CD16+ (macrophages), CD19+ (B lymphocytes), and CD40+, CD80+, and CD86+ (costimulatory molecules on macrophages, dendritic cells and B lymphocytes), and an increase in CD4+IL-10 cells, is expected. Further, osteogenic differentiation by cells in vitro is anticipated and has been documented (as shown in FIG. 2).

Aim 2: Evaluate Functional Bone and Cartilage Regeneration by Xenogeneic Transplant Cells.

Aim 2 is designed to determine that engineered PDL1+ xenogeneic cells will accelerate bone and cartilage regeneration equal to syngeneic cells in an NIH mu athymic nude rat osteotomy model [as described in Zachos, T. et al., MSC-mediated gene delivery of BMP2 in articular fracture model, Mol Ther (2007) 15:1543, the disclosure of which is incorporated by reference herein in its entirety]. The nude rat is a cost effective in vivo model that is immunosuppressed in order to be able to evaluate human genes and cells without an immune reaction. The rat is also of sufficient size to permit joint surgery and imaging techniques of high resolution. [Zachos, T. A. et al., Rodent Models for the Study of Articular Fracture Healing, J Investigative Surgery (2007) 20:87-95]. The female gender is consistent and a young age ensures rapid bone and cartilage regeneration. Aim 2 is further designed to determine that native xenogeneic cells will be superior to saline, but inferior to syngeneic cells, thereby quantifying a biologic benefit of xenogeneic cells despite an immune reaction.

Genetically engineered syngeneic cells have been demonstrated to accelerate bone and cartilage regeneration in vivo using an articular fracture rat model [described in Zachos, T. et al., MSC-mediated gene delivery of BMP2 in articular fracture model, Mol Ther (2007) 15:1543, incorporated by reference herein in its entirety]. This model uses an immunodepressed rat (NIH rnu athymic nude rat) permitting adoptive transfer [Yuan R. et al., D103+CD8+ effectors promote tubular injury following allogeneic renal transplantation, J of Immunol (2005) 175:2868, incorporated by reference herein in its entirety] of human lymphocytes to establish a human/rat chimera.

Adoptive transfer of allogeneic lymphocytes to assess immune reaction between donor and recipient in vivo is a well established tool, well known to those of ordinary skill in the art, in transplantation research [as described in Yuan R. et al., D103+CD8+ effectors promote tubular injury following allogeneic renal transplantation, J of Immunol (2005) 175:2868, incorporated by reference herein in its entirety]. The human/rat chimera will permit the biologic interaction of human T-cells with implanted xenogeneic equine cells during the process of fracture repair. Rapid immune rejection and death of the xenogeneic transplant cells is anticipated, thereby preventing the accelerated repair produced by positive control syngeneic cells engineered to express hBMP2. It is anticipated that PDL1-induced immunotolerance of human immune cells to the xenogeneic cells may permit fracture regeneration equal to those positive control cells. It is also possible that xenogeneic cell survival is sufficient to permit acceleration of fracture repair despite an immune reaction. Use of the in vivo model described herein will permit the quantification of these multiple possible outcomes as well as the ex vivo measurement of the human immune response to the xenogeneic cells by synovial culture after fracture harvest.

Aim 2a: Quantify the Function of Xenogeneic Cells to Accelerate Bone and Cartilage Regeneration.

Referring to FIGS. 5 and 6, the “best” xenogeneic cell source identified in Aim 1 will be selected for use in vivo. “Best” xenogeneic cells will be cultured and processed [as previously published in Zachos, T. et al., MSC-mediated gene delivery of BMP2 in articular fracture model, Mol Ther (2007) 15:1543, incorporated by reference herein in its entirety] for injection into the articular femoral condylar osteotomy, which will have been surgically created three days earlier in the human/rat chimeras. The experimental test groups will include native (PDL1−) xenogeneic cells and PDL1+ xenogeneic cells, with each being compared to (1) vector control xenogeneic cells (GFP+); (2) no cells (saline); and (3) engineered syngeneic cells (positive control). Outcomes evaluated on Day 24 will include amount and density of bone within the fracture gap measured by quantitative micro-computed tomography (pCT) and histomorphometry (% bone filling the fracture gap) (techniques well know to those of ordinary skill in the art), and quality of articular cartilage over the fracture gap (i.e., the number of chondrocyte lacunae and histochemical staining intensity of cartilage matrix). At the termination of the study (Day 24), knee joint synovium will be cultured and FACS analysis performed as in Aim 1 to determine the profile of human immune cells. Sorted CD45+ human cells from the synovium will be re-co-cultured with the original donor cells to quantify effector T-cell killing of donor cells as in Aim 1. The results will determine the efficacy of engineered xenogeneic cells to accelerate bone and articular cartilage regeneration, as well as characterize the human immune response to xenogeneic cells in vivo.

In general, cells (eqMSC, eqC, or eqDfb) will be studied using seven groups (Gp) of 10 animals (See Table 2). As shown in Table 2, six groups of rats (Gps 2-7) will be injected subcutaneously on Day 0 with 5×10⁷ human splenocytes over the lateral tibia area, distal to the knee joint. On Day 7, the articular femoral osteotomy will be created and injected percutaneously three days later with 100 μl of saline (Gey's balanced salt solution, commercially available from Gibco, Grand Island, N.Y.) [saline chimera control], syngeneic rat cells producing the osteogenic molecule hBMP2 [prepared as published in Zachos, T. et al., MSC-mediated gene delivery of BMP2 in articular fracture model, Mol Ther (2007) 15:1543] (5×10⁶ cells/100 μl saline; positive controls; Gps 1 and 2), native (PDL1−) xenogeneic cells (5×10⁶ cells/100 μl saline), xenogeneic expressing hPDL1 (5×10⁶ cells/100 μl saline), xenogeneic co-expressing hBMP2 and GFP (5×10⁶ cells/100 μl saline; vector control) or xenogeneic co-expressing hBMP2 and hPDL1 (5×10⁶ cells/100 μl saline) (See Table 2 and FIG. 5). Biologic function of the cells to accelerate bone and cartilage regeneration will be quantified by pCT and histomorphometry, as is well known to those of ordinary skill in the art, at 24 days after injection.

TABLE 2
Rat Osteotomy Injection Assignments
Rat Group (n = 10/group)

1. Syngeneic MSC-hBMP2 (positive control)
2. Syngeneic MSC-hBMP2 Chimera (chimera control)
3. Saline Chimera (negative control)
4. Xenogeneic “Best” Cell Chimera
5. Xenogeneic “Best” Cell-PDL1 Chimera
6. Xenogeneic “Best” Cell-BMP2/GFP Chimera
7. Xenogeneic “Best” Cell-BMP2/PDL1 Chimera

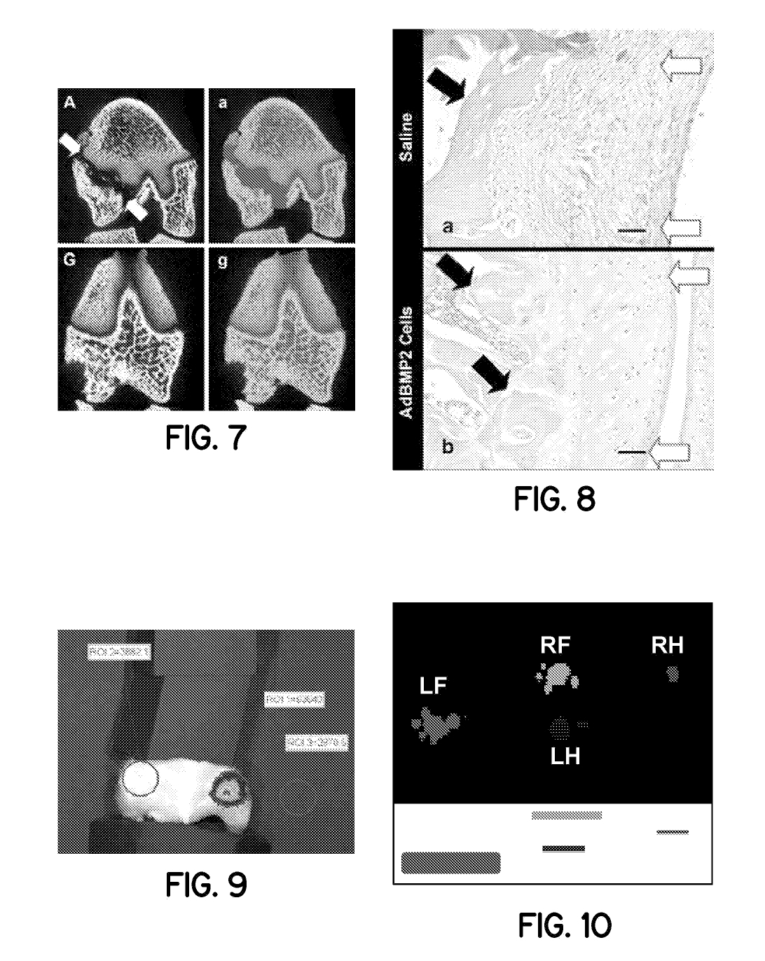

Referring to Table 2 and FIG. 7, outcomes will be checked for normality, then analyzed using the mixed effects linear regression model and assumptions described for Aim 1 (a error 0.05 and a power of 0.8) for time (repeated Days) and Injection Assignment (Table 2). Using the most variable outcome in published studies using the same animal model, (mean saline injected gap area 1987 pixels +/−1000), N=10 subjects would detect a 50% bone regeneration in the osteotomy gap. If a treatment improves bone regeneration <50%, this will be deemed an efficacy that does not warrant continued investigation, at least without modification.

More specifically, regarding the articular fracture rat model and assignments, seventy female NIH rnu athymic nude rats (10-12 weeks of age) (available from Charles River Laboratories, Wilmington, MA) will be assigned to seven groups of ten rats each (see Table 2). On Day 0, six groups of rats will receive a subcutaneous injection of 5×10⁶ human mixed splenocytes (each of the 10 humans represented in each group of rats) distal to the knee joint over the lateral side of the tibia to induce a rat/human immune cell chimera (chimera) (see FIGS. 5 and 6). On Day 7, all rats will be pre-medicated with meloxicam (Metacam®, Boehringer Ingelheim Vetmedica, Inc. St. Joseph, Mo., 5.0 mg/kg) and butorphanol tartrate (Torbugesic®, Wyeth, Madison, N.J., 2 mg/kg) and general anesthesia induced and maintained using 3% isoflurane in 100% oxygen. A lateral intercondylar osteotomy will be performed via lateral parapatellar arthrotomy of the stifle joint using 2.5× surgical magnifying loupes [as described in Zachos, T. et al., MSC-mediated gene delivery of BMP2 in articular fracture model, Mol Ther (2007) 15:1543; and Zachos, T. A. et al., Rodent Models for the Study of Articular Fracture Healing, J Investigative Surgery (2007) 20:87, incorporated by reference herein in their entireties] (see FIG. 5). This model induces cartilage and bone injury to mimic articular fracture, but maintains a relatively stable joint because the cruciate ligaments, collateral ligaments and meniscal ligaments are undisturbed. Animals will weightbear after surgery [Zachos, T. et al., MSC-mediated gene delivery of BMP2 in articular fracture model, Mol Ther (2007) 15:1543; Zachos, T. A. et al., Rodent Models for the Study of Articular Fracture Healing, J Investigative Surgery (2007) 20:87].

On Day 10, rats will receive the assignments in Table 2. Groups receiving “best” xenogeneic cells [MSC, C, or Dfb], will have cells cultured in monolayer three days before surgery, then be detached with EDTA-Trypsin and subjected to centrifugation at 450 g for 10 minutes at room temperature (see FIG. 6). Cells will be suspended in saline solution (Gey's balanced salt solution, Gibco, Grand Island, N.Y.) at a concentration of 5×10⁷ cells/mL for injection. The fracture will be injected percutaneously with 5×10⁶ xenogeneic cells in 100 μL of saline solution into the articular fracture gap. Xenogeneic cells to express GFP or PDL1 will be transduced with either scAAV2-RGD-PDL1 or scAAV-RGD-GFP at the MOI anticipated to be 1×10⁵ DNA particles/cell based on pilot work (FIG. 4). This vector dose is expected to generate gene expression visible by the GFP expression within two days. Previous data confirmed the injection goes into the joint as well as the fracture, and thereby is expected to expose the human immune cells in and around the joint to xenogeneic cells. Six groups of rats will receive the subcutaneous injection of human splenocytes (5×10⁶ cells in 100 μL) at the start of the study (as shown in Table 2). Rats not receiving human splenocytes (Gp1) will serve as the true positive control of bone regeneration using this model. These rats will receive the syngeneic MSC engineered to express hBMP2. This treatment has been shown to regenerate bone and articular cartilage within 14 days after injection (Day 24 of the study) [see Zachos, T. et al., MSC-mediated gene delivery of BMP2 in articular fracture model, Mol Ther (2007) 15:1543, incorporated by reference herein in its entirety]. In that study, osteotomy healing in this positive control group occurred in all rats (n=7) and within days, producing a reliable model of genetically engineered acceleration of bone and cartilage regeneration (see FIG. 7). Importantly, both bone and cartilage regenerated in this BMP2 model (see FIG. 8).

Tracking of Human Splenocytes and Xenogeneic Transplant Cells: One rat of ten from each of the six groups to receive human splenocytes (Table 2; Gp 2-7) will have splenocyte cultures transduced with firefly luciferase gene encoded by an adenoviral vector (Ad-Luc) (MOI 1×10² infectious particle) [Zachos, T. et al., Gene-mediated osteogenic differentiation of stem cells by BMP-2 or -6, J Orthop Res (2006) 24:1279; Ishihara, A. et al., Bone healing enhanced by autologous dermal fibroblasts expressing BMP2, 55th ORS (2009) 840] added to the well for two hours at 24 hours prior to injection. A quadruplicate well will be imaged [IVIS; Xenogen Corp., Alameda, Calif.] to confirm luciferase expression prior to injection. In vivo imaging of the 6 rats, under general anesthesia, of both knee joints will be performed on Day 0 (before injection of Ad-Luc splenocytes) and on days 1, 2, 3, 5 and 7. Subsequently, a different rat from each of the 6 groups to receive syngeneic or xenogeneic cells (Table 2) will have xenogeneic cultures transduced with Ad-Luc as described above for splenocytes. In vivo imaging, under general anesthesia, of both knee joints will be performed on Day 10 (before injection of Ad-Luc xenogeneic cells) and on days 11, 12 and13 and then every other day until no detection.

Aim 2b: Quantify in Vivo Immunotolerance to Xenogeneic Cells.

Immunotolerance in vivo to the xenogeneic cells will be quantified by ex vivo synovium culture, representing rat and human cells and analysis as in Aim 1b. FACS sorted CD45+ human immune cells from the synovium will be challenged in vitro with xenogeneic cells (second exposure) and analyzed as stated in Aim 1b for acute inflammatory reaction, T-cell proliferation, and effector cell killing (CTL activation).

In Vivo Outcome Assessments: On Days 10 and 24 (prior to and 14 days after injection of xenogeneic cells), kinetic gait analysis will be performed as an objective and quantitative technique to assess functional limb use [Noldus CatWalk® XT System]. This apparatus is designed for laboratory animals. Video capture trans-illuminates footprints to digitize and calculate dynamic and static gait parameters, including stride length, maximal contact duration, and print magnitude (area), which represents weight distribution to each paw. Use of CatWalk® has been previously validated for use in guinea pigs both prone and resistant to osteoarthritis to identify parameters critical for comparison (see FIG. 10). This clinically relevant, and vitally important, outcome measure for successful acceleration of bone regeneration is not known to have been reported for rodents. However, those of ordinary skill in the art are experienced with kinetic gait analysis with large animal species [see Ishihara, A. et al., Kinetic gait analysis for the detection, quantification, and differentiation of lameness and spinal ataxia: 36 horses, J Am Vet Med Assoc (2009) 234 (5):644, incorporated by reference herein in its entirety].

On Day 24, animals will be euthanized by isoflurane inhalation followed by cervical dislocation. This method of euthanasia is used to avoid injecting drugs that could affect the requirement for live, healthy cell harvests. The AVMA guidelines on euthanasia are met with these methods. Osteotomized limbs, and the contralateral knee joint, will be immediately harvested. The soft tissues of the knee joint (synovium and joint capsule) will be carefully removed aseptically from under a dissecting microscope for cell isolation and culture. Articular soft tissues are expected to contain human and rat cells. Cultured synovial cells from the chimeric rats will be processed for FACS analysis for the complete panel of tests described in Aims 1 and 2 and as shown in FIG. 4. CD45+ human cells will be sorted, processed, and re-cultured for re-exposure to original donor xenogeneic cells to evaluate delayed CMI to xenogeneic cells as described for both Aims 1 and 2.

Seventy rat osteotomized knee joints will be maintained at 4° C. and scanned in air using a Siemens Inveon™ imaging system (Siemens, Erlangen Germany) at 30 mm voxel resolution. The bone mineral density (BMD, mg/cc) of each specimen will be determined by calibrating the images using hydroxyapatite standards, defining a region of interest completely contained within the osteotomy gap, and setting an appropriate threshold level for bone using Visage™ volume visualization software. This software will then be used to trace the exact outline of the osteotomy gap in each specimen and to calculate total osteotomy gap area and volume from two-dimensional images in the axial (transverse), coronal, and sagittal computed tomographic imaging planes.

A region of interest will be selected entirely within the osteotomy gap (if present) in each femur, and mean grayscale value, bone volume of the same selected region within the osteotomy gap, voxel values of the region, bone mineral content, bone mineral density, tissue mineral content, tissue mineral density, and bone volume fraction of the gap will be calculated. Subsequently and within 24 hours, knees will be fixed in 10% neutral buffered formalin for 72 hours at room temperature, decalcified in 10% EDTA (pH 7.4) for seven days, sectioned at 6 um, and stained with hematoxylin and eosin and safranin 0 and fast green. Sections will semi-quantitatively evaluated for bone and cartilage quality by the PI with a board certified pathologist [Zachos, T. et al., MSC-mediated gene delivery of BMP2 in articular fracture model, Mol Ther (2007) 15:1543].

Handling of rats will always occur under general anesthesia. Most rats (n=57) will be handled four times, once for splenocyte injection, once for surgery, once for cell injections, and one at termination of the study. A subset of rats (n=13) will be anesthetized daily for ˜4-7 days after one of the injections for IVIS imaging. For surgery, all rats will be pre-medicated with meloxicam (Metacam®, Boehringer Ingelheim Vetmedica, Inc. St. Joseph, Mo., 5.0 mg/kg) and butorphanol tartrate (Torbugesic®, Wyeth, Madison, N.J., 2 mg/kg) for pain management and general anesthesia will be induced and maintained using 3% isoflurane in 100% oxygen, a routine technique even for clinical veterinary patients. This model induces cartilage and bone injury to mimic articular fracture, but maintains a relatively stable joint because the cruciate ligaments, collateral ligaments and meniscal ligaments are undisturbed. Animals will weightbear after surgery. For each injection and at termination, all animals will be anesthetized with inhalant isoflurane in a belle jar.

Statistical analysis for Aim 2: Quantitative outcomes [Days 14 and 24 osteotomy limb stride duration, magnitude, length; Day 24 osteotomy gap area, bone volume, bone mineral content, bone mineral density, lacunae number, chondrocyte number, etc.] will be checked for normality, then analyzed using the mixed effects linear regression model and assumptions described for Aim 1 for time (repeated Days) and Injection Assignment (Table 2). Human splenocyte source will serve as a random effect. Using the most variable outcome in published studies using the same animal model [see Zachos, T. et al., MSC-mediated gene delivery of BMP2 in articular fracture model, Mol Ther (2007) 15:1543, incorporated by reference herein in its entirety], (i.e. mean saline injected gap area 1987 pixels+/−1000), N=10 subjects would detect a 50% bone regeneration in the osteotomy gap. If a treatment improves bone regeneration <50% this will be deemed an efficacy that does not warrant continued investigation, at least without modification.

In summary, it is anticipated that syngeneic cells expressing BMP2 will be potently osteogenic in this rat model as previously published [Zachos, T. et al., MSC-mediated gene delivery of BMP2 in articular fracture model, Mol Ther (2007) 15:1543, incorporated by reference herein in its entirety]. Further, the selected xenogeneic cells will induce human T-cell activation in vivo in this model and this will be inhibited by PDL1 expression on the cells. It is anticipated that this will translate into inferior bone and cartilage regeneration with native xenogeneic cells as compared to syngeneic cells. Further, it is anticipated that PDL1+ xenogeneic cells will be equivalent to syngeneic cells in the acceleration of bone and cartilage regeneration in this model. Xenogeneic cells expressing hBMP2 and not expressing PDL1 (PDL1− cells) may significantly accelerate osteotomy regeneration compared to saline or native xenogeneic cells without hBMP2 (Gp 4), indicating a threshold for clinical tolerance and efficacy to xenogeneic immune reaction.

### Example 2

This Example is directed to transduction of PDL-1 by AAV vectors. Such information was discussed above in prophetic Example 1, and the results of transduction using AAV vectors is described in this Example 2. In particular, as described in greater detail below, AAV vectors are used to efficiently transduce equine cells and then will be evaluated for gene expression via western blot and also via PCR analysis.

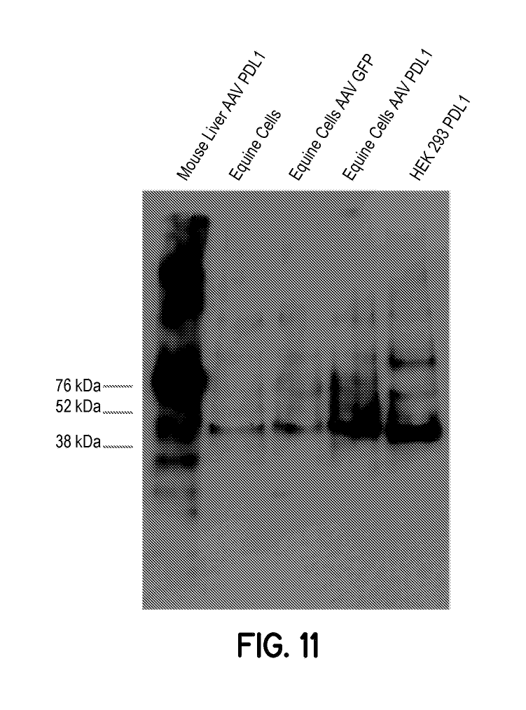

Referring to FIG. 11, endogenous equine (eq) PDL1 is expressed (light band at ˜48 KDa) from equine cultured cells determined by western blot. Equine PDL1 protein is similar in size to murine (m) PDL1 overexpressed in equine cells and HEK cells using the AAV8.U1a. mPDL1 vector. These studies confirm the ability of the AAV8 vector construct to effectively transduce equine cells and overexpress PDL1. Antibody reagents known to work with murine cells, cross react with equine and human cells.

The methods used in obtaining the results discussed above and shown in FIG. 11 are as follows: Total protein extracts from both tissue and cultured cells were run on 8-10% acrylamide gel and Nitrocellulose membrane using a semi-dry transfer method (known to those of ordinary skill in the art) and 5% non-fat milk in TTBS as a blocking solution.

The first antibody used was anti-mouse B7-H1 antibody. (R&D MAB1019). The dilution was 0.5-2 ug/ml (titrate every new lot), and incubation was for 6 hours at room temperature, or overnight at 4° C.

The second antibody was anti-rat-HRP. (Sigma A5795). The dilution was 1:5,000, and incubation was for 45 minutes at room temperature.

In reviewing the bands shown in FIG. 11: When looking at the Equine Mesenchymal Stem Cell Controls, those untreated and treated with AAV2-GFP show endogenous expression at ˜48 KDa. The Equine Cells and HEK293 Cells. Infected with AAV8.U1a. mPDL1 show overexpression (strong band) at ˜48 KDa.

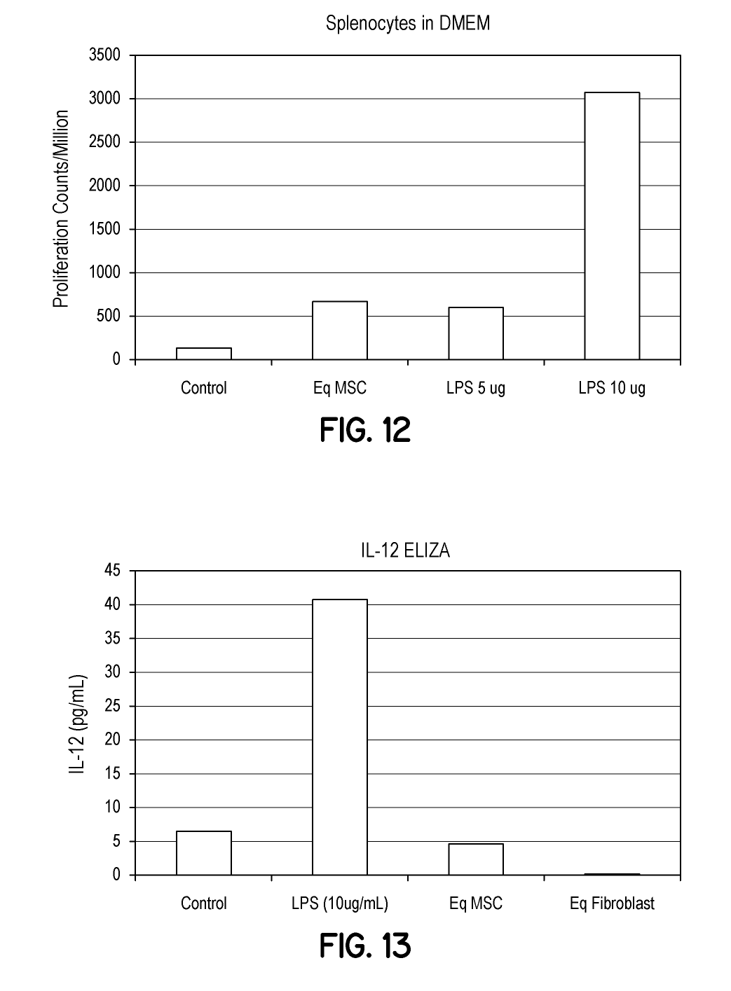

Further, a tritiated thymidine proliferation assay confirmed equine MSC co-cultured with murine splenocytes induced splenocyte proliferation (as reported with the CSFE assay in the proposal) similar to low dose LPS validating the CSFE assay and a xenogeneic reaction (see FIG. 12).

Further, purchased Human PDL1 [Open Biosystems; pCR4-TOPO vector] was cut and ligated into the AAV gene expression plasmid containing the CMV promoter [pHpa-tris-KS gene expression vector—obtained from Dr. Doug McCarty, The Ohio State University]. AAV plasmid [0.8 ug DNA] was transfected into equine cells in monolayer culture at 90% confluence using lipofectamine 2000 [11668-027; Invitrogen; Carlsbad, Calif.]. Cells were harvested 18 hrs after transfection and RNA extracted with TRIZOL [15596-018; Invitrogen; Carlsbad, Calif.]. Human PDL1 gene expression was confirmed by primers designed from our sequencing data of the human PDL1 from the TOPO vector. The forward primer is: GGGCCCGGCTGTTGAAGGAC [SEQ. ID NO. 4] [Applied Biosystems], and the reverse primer is: AGCGGTACACCCCTGCATCCT [SEQ. ID NO. 5] [Applied Biosystems].

Human PDL1 gene expression was detected in transfected equine cells and not in untransfected equine cells by RT-PCR [Applied Biosystems; Sybr Green] as described in the proposal. These data suggest the primers may be specific for the human sequence.

### Example 3

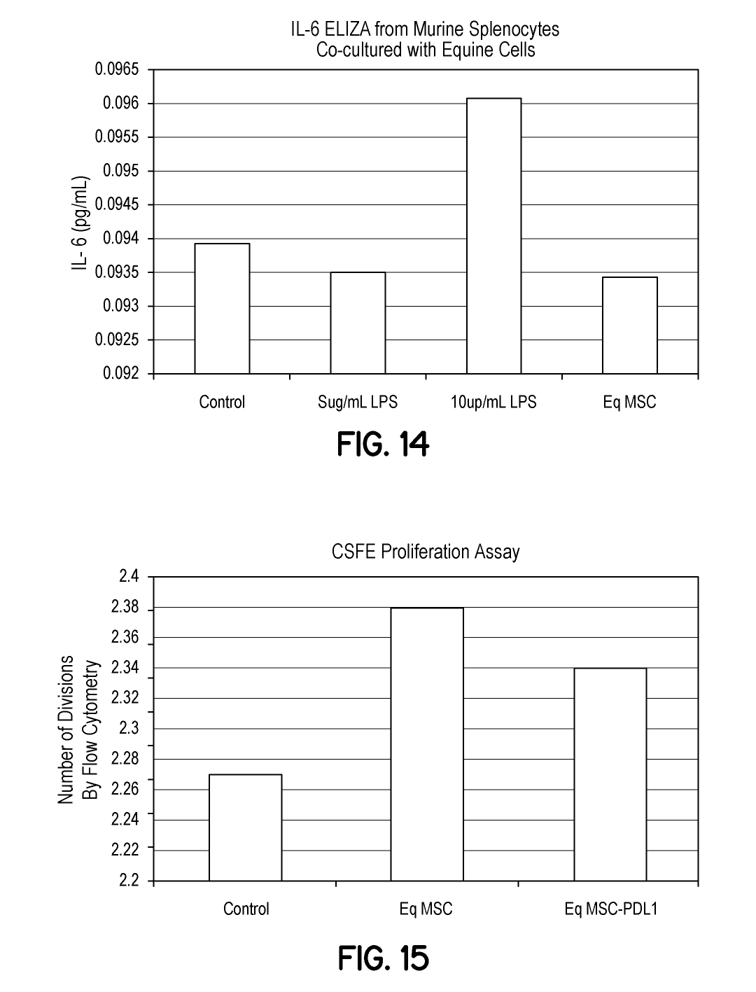

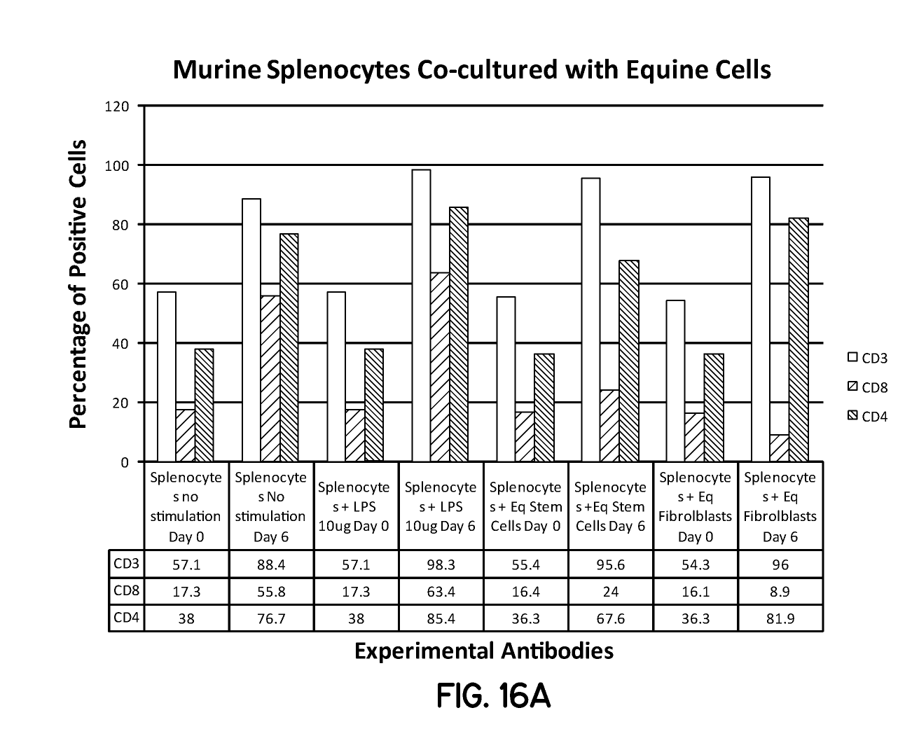

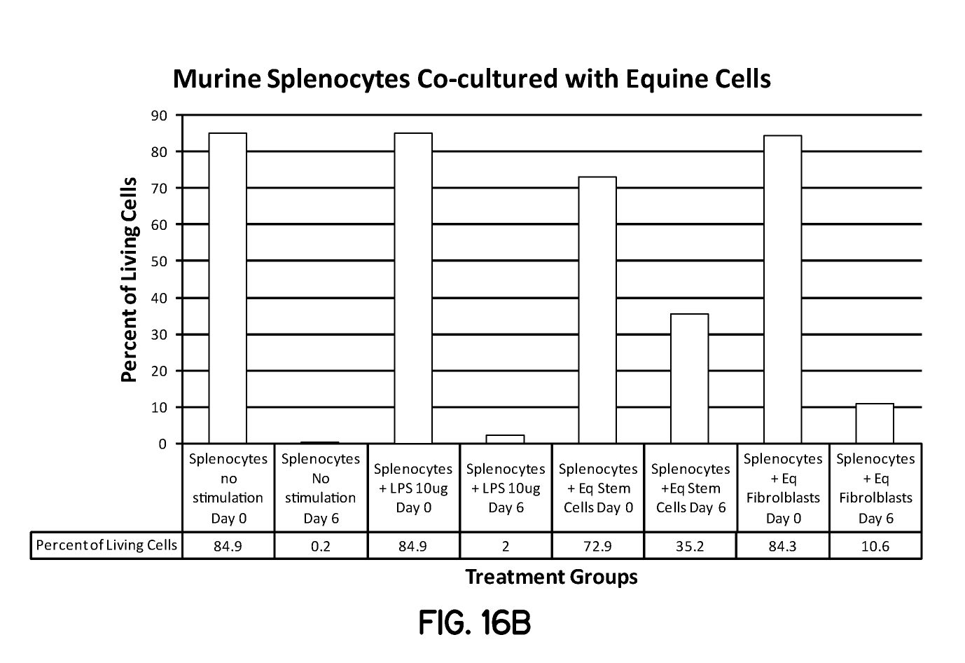

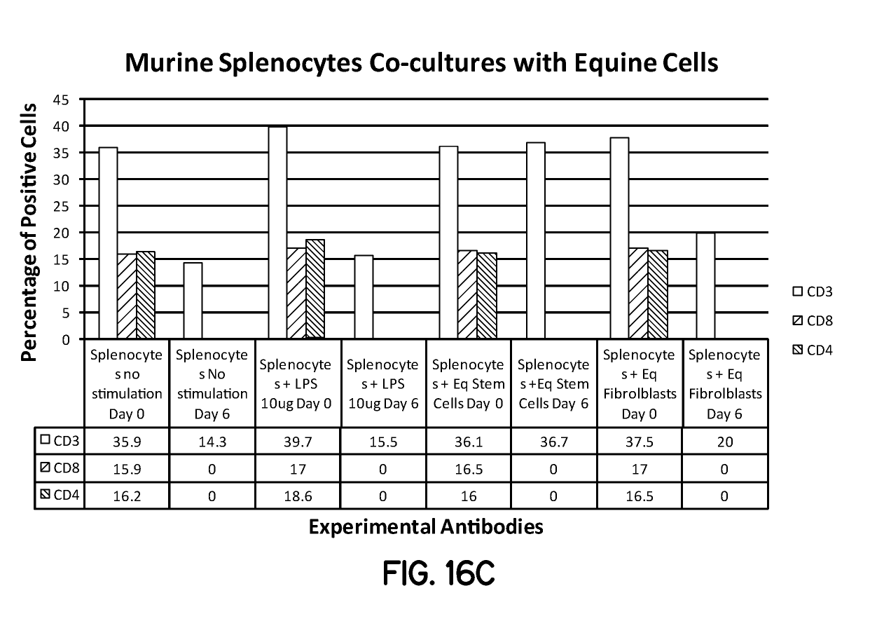

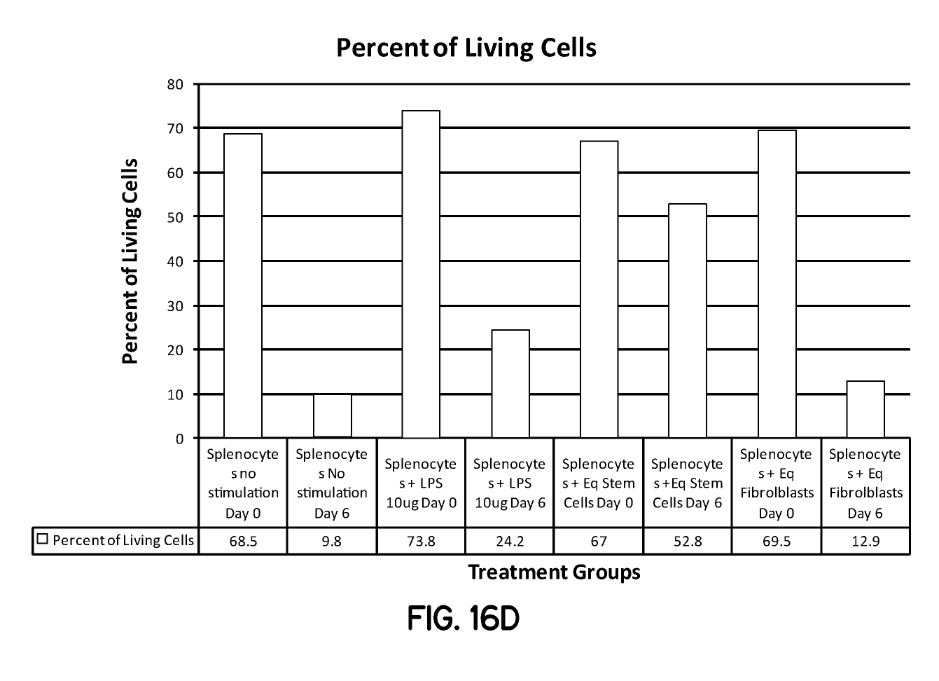

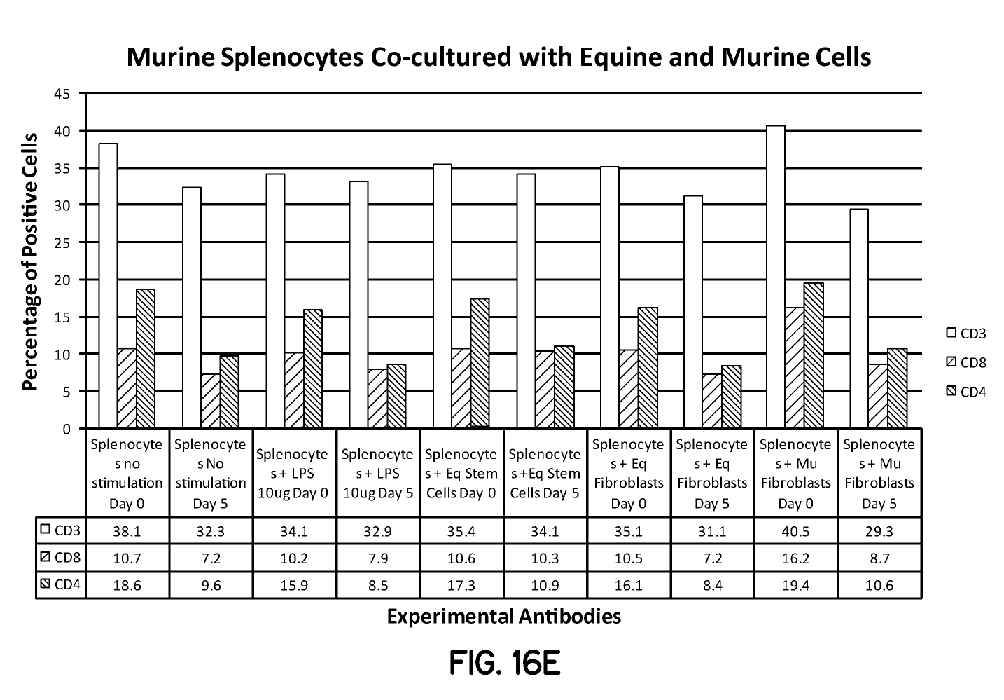

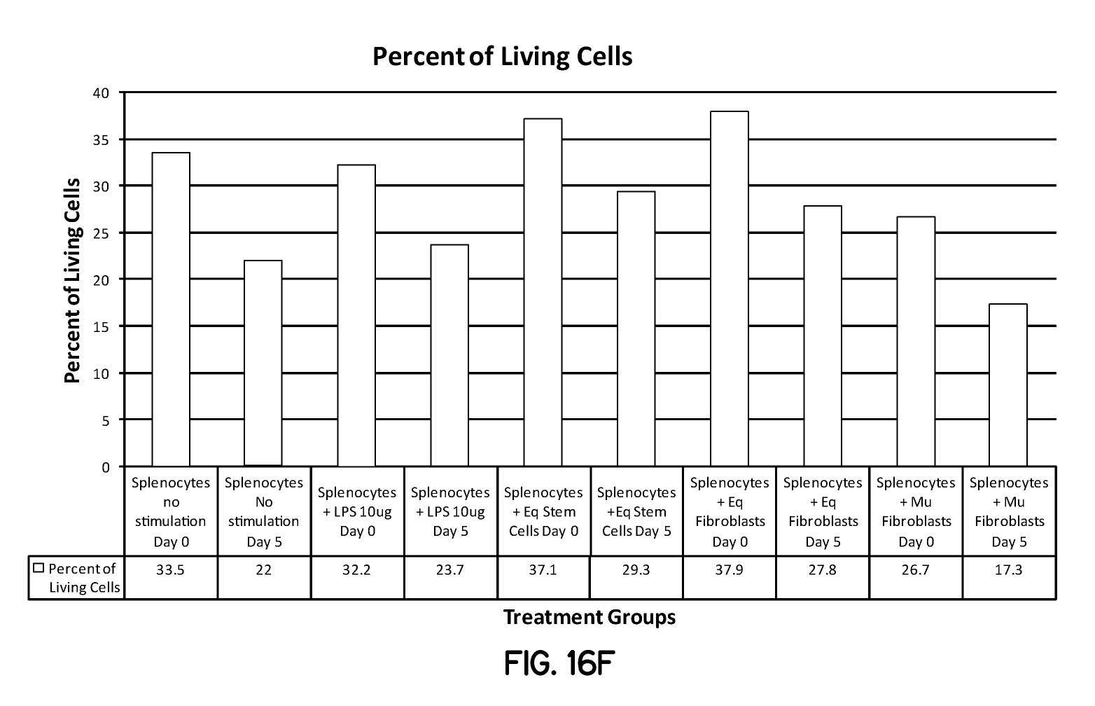

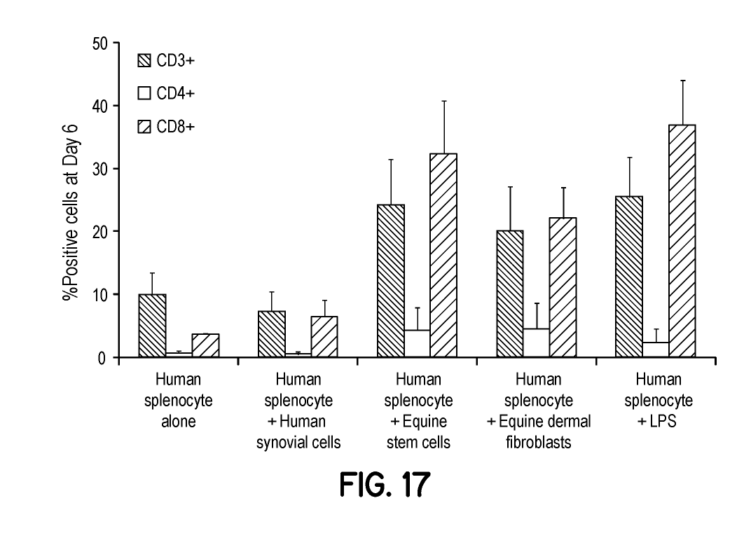

This Example is directed to studies using xenogeneic co-cultures of murine splenocytes with equine stem cells or equine fibroblasts to study the interaction between these xenogeneic cells. We have also studied co-cultures of human splenocytes with equine stem cells or equine fibroblasts. The findings, detailed below and as shown in FIGS. 13-17, suggest that immune cells (murine and human) increase in splenocyte cultures when xenogeneic cells are in contact with them. However, our data also suggests that splenocyte cultures (as performed with murine cultures) live longer and do not die out when cultured with equine stem cells or fibroblasts and the effect seemed greater with equine stem cells (as compared to fibroblasts). The graphs shown in FIGS. 13-17 detail the various cells that are co-cultured with one another (e.g., murine splenocytes, equine stem cells, equine fibroblasts). Parameters for co-culture (e.g., media, temperature, time, etc.) are well known to those of ordinary skill in the art, and can be achieved without undue experimentation. As a non-limiting example, some co-culture data is given above in prophetic Example 1.

Based on these studies, the conclusion is that xenogeneic cultures have a trophic influence on splenocytes (immune cells). This trophic effect was observed in co cultures in which the murine splenocytes were in physical contact with the equine cells and was observed in co-cultures in which the cells are kept physically separate but the media or fluid baths both the murine splenocytes and the equine cells. This suggests there is both a contact and soluble factor influence of xenogeneic interaction that is supportive to immune cell robustness and health. The murine splenocyte/murine fibroblast co-cultures shown died out earlier than the xenogeneic co-cultures. The murine splenocyte/equine cells co-culture data also suggests that acute inflammation is not largely activated, as seen by the IL-12, INF and IL-6 data.

So, in summary, the conclusion is that xenogeneic (human and murine host/ equine donor) interactions will elicit an immune cell (CD+) response that may be induced to tolerance by expressing PDL-1 on the equine donor cell. A further conclusion is that xenogeneic interaction may provide a separate benefit that generally supports the health of the host immune system cells.

As various changes could be made in the above-described aspects and exemplary embodiments without departing from the scope of the invention, it is intended that all matter contained in the above description shall be interpreted as illustrative and not in a limiting sense.

What is claimed is:

1. A transplant system, comprising:
at least one isolated cell from a xenogeneic donor, the at least one cell to be transplanted into a host, wherein the at least one cell is adapted to include a gene that causes expression of a protein that enhances orthopedic healing.

2. The transplant system of claim 1, wherein the protein is chosen from PDL1, BMP2, CTLA4, and combinations thereof.

3. The transplant system of claim 1, wherein the xenogeneic donor is a horse.

4. The transplant system of claim 1, wherein the host is chosen from a rat and a human.

5. The transplant system of claim 1, wherein the at least one cell is chosen from a mesenchymal stem cell, a chondrocyte, and a dermal fibroblast.

6. The transplant system of claim 1, wherein the at least one cell is an equine cell engineered via a scAAV mutant vector to cause expression of hPDL1.

7. A method of regenerating tissue, the method comprising:
engineering at least one isolated cell from a xenogeneic donor to cause the at least one cell to express a protein that enhances orthopedic healing; and
transplanting the at least one isolated cell from the xenogeneic donor into a host.

8. The method of claim 7, wherein the protein is chosen from PDL1, BMP2, CTLA4, and combinations thereof.

9. The method of claim 7, wherein the xenogeneic donor is a horse.

10. The method of claim 7, wherein the host is chosen from a rat and a human.

11. The method of claim 7, wherein the at least one cell is chosen from a mesenchymal stem cell, a chondrocyte, and a dermal fibroblast.

12. The method of claim 7, wherein the at least one cell is an equine cell engineered via a scAAV mutant vector to cause expression of hPDL1.


SEQUENCE LISTING (5 sequences)

SEQ ID NO: 1 - DNA, 20 bp, Artificial Sequence
  Synthetic
gccgaagtcatctggacaag

SEQ ID NO: 2 - DNA, 20 bp, Artificial Sequence
  Synthetic
tctcagtgtgctggtcacat

SEQ ID NO: 3 - DNA, 20 bp, Artificial Sequence
  Synthetic
caccaccaccaattccaaga

SEQ ID NO: 4 - DNA, 20 bp, Homo sapiens
gggcccggctgttgaaggac

SEQ ID NO: 5 - DNA, 21 bp, Homo sapiens
agcggtacacccctgcatcct